(19) United States
(12) Patent Application Publication
(10) Pub. No.: US 2026/0166105 A1
(43) Pub. Date: Jun. 18, 2026

(54) Multifunctional Plant-Based Supplement Compositions and Related Methods

(71) Applicant: Maureen A. McKenzie, Homer, AK (US)

(72) Inventor: Maureen A. McKenzie, Homer, AK (US)

(21) Appl. No.: 19/080,781

(22) Filed: Mar. 14, 2025

Related U.S. Application Data
(60) Provisional application No. 63/565,516, filed on Mar. 14, 2024.
(60) Provisional application No. 63/567,426, filed on Mar. 20, 2024.

(51) Int. Cl. A61K 36/45; A61K 31/122; A61K 31/352; A61K 31/353; A61K 31/357; A61K 31/366; A61K 31/565; A61K 36/185; A61K 36/28; A61K 36/288; A61K 36/738; A61K 47/22; A61K 47/24; A61P 7/02; A61P 17/02; A61P 35/00; A61P 35/02

(52) U.S. Cl. CPC A61K 36/45; A61K 31/122; A61K 31/352; A61K 31/353; A61K 31/357; A61K 31/366; A61K 31/565; A61K 36/185; A61K 36/28; A61K 36/288; A61K 36/738; A61K 47/22; A61K 47/24; A61P 7/02; A61P 17/02; A61P 35/00; A61P 35/02; A61K 2236/15; A61K 2236/17

(57) ABSTRACT

A dietary supplement composition provides a multifaceted beneficial effect, including antioxidant, anti-inflammatory, wound healing, and anticancer activities. The composition comprises a blend of plant-based concentrates derived from Vaccinium, Rosa, Taraxacum, and Achillea species. It delivers a broad spectrum of phytochemicals, including polyphenolics, flavonoids, and terpenoids, which are known for their health-promoting properties. Such a composition may optionally be enhanced by adding astaxanthin, and/or birch bark extract containing betulinic acid, and/or other bioactive agents. The supplement is desirably prepared by methods that ensure stability and bioavailability of the phytochemicals, including cold processing of plant materials, and controlled drying techniques. It offers a natural, plant-based approach to supporting human and animal health, with potential applications extending to the maintenance of wellness and the adjunctive treatment of various health conditions.

### CROSS-REFERENCE TO RELATED APPLICATIONS

This application claims priority benefit of U.S. Provisional Patent Application 63/565,516 filed Mar. 14, 2024, and U.S. Provisional Patent Application 63/567,426 filed Mar. 19, 2024, the disclosures of which are hereby incorporated by reference to the extent not inconsistent with the present disclosure.

### TECHNICAL FIELD

The present invention relates to formulations of supplements with a wide range of activity, produced from dried plant-based components.

### SUMMARY OF THE INVENTION

The present invention provides a novel combination of botanical concentrates to improve quality of life. A core matrix for various supplement formulations consists of four whole food or medicinal plant concentrates that together deliver virtually every class of bioactive phytochemical. (CoreMatrix is a trademark of DENALI BioTechnologies, Inc. [Homer, Alaska] for dietary and nutritional supplements.) The formulations provide the basis of multifunctional supplements that may optionally be provided with antioxidant, anti-inflammatory, wound healing, and anticancer activities. This is pertinent to any stressful condition such as aging, cardiovascular disease, diabetic complications, surgical recovery (for any reason), and cancer as an adjunct therapeutic agent. Since every major phytochemical class occurs in the four concentrates, it serves as an appropriate natural matrix for any phytochemical addition. Addition of certain extracts and purified compounds to the core matrix is dictated by the targeted disease or condition. The core matrix-containing supplement may be administered to humans and other mammals, including dogs and horses.

The four basic ingredients of the core matrix are Vaccinium, Rosa, Taraxacum, and Achillea. Inherent, powerful, and broad antioxidant activity is enhanced by the addition of astaxanthin. Antioxidant and anti-inflammatory activity are further enhanced by those five plus birch bark preparations. Antioxidant activity is the foundation for anti-inflammatory activity and the plethora of associated effects. These effects are intimately and, at times paradoxically, involved in wound healing but in ways best observed empirically in vivo. This is even more applicable to cancer in which numerous interactions are extremely complex, sometimes counter-intuitive, and unpredictable.

Vaccinium Concentrate (VC) is a unique ingredient, with respect to phytochemical profile, that offers exceptional antioxidant activity in vitro and in cellular model systems; noted anti-aging/survival activities; unusual activity against pro-inflammatory cytokines, CCL2 and CXCL5; wound healing capacity, particularly in Phase 1 with platelets, and potential application in many cancers.

Likewise, Rosa Concentrate (RC) is phytochemically unique, has potent antioxidant activity, and has numerous components with in vitro tumor cytotoxicity. As a component of the core matrix of the present invention, RC complements VC.

Taraxacum Concentrate (TC) and Achillea Concentrate (AC) are included in formulations of the present invention to round out the phytochemical profile of the formulation.

In certain applications, a birch bark powder/purified extract is included in the formulation as an addition to the core matrix. This may be used as a dietary supplement or basis for delivery of specific phytochemicals directed against disease-related molecular targets.

The beneficial results mentioned above are conferred by the combination of Vaccinium Concentrate (VC), Rosa Concentrate (RC), Taraxacum Concentrate (TC), Achillea Concentrate (AC) that provide a matrix for stability and bioaccessibility/bioavailability of various other ingredients, each with bioactivities of their own. This combination provides a matrix of components for every class of phytochemical bioactive, unachievable by others without employing typically 15-30 whole fruit/herb constituents. Notably, VC offers the broadest range of polyphenolics and exclusively anthocyanins, RC offers phenolic acids, catechins, and lycopene, and TC and AC other carotenoids, various terpenoids, alkaloids, and numerous other phytochemical classes.

In preferred embodiments of the invention, source plants from Alaska are used due to their exceptional phytochemistry. This is preferably preserved by utilizing Refractance Window Drying (RWD), as by far the best commercially available drying method. See e.g. (Raghavi, L M et al. J. Food Engineering. 2018, 222, 267. doi:10.1016/j.jfoodeng.2017.11.032) RWD equipment is available from commercial sources including GEM Machinery & Allied Industries, Village-Biprannapara, Jungalpur, India.

### BRIEF DESCRIPTION OF THE DRAWINGS

FIG. 1 is a depiction of major classes of phytochemicals.

FIG. 2A is a graph of reverse phase high-performance liquid chromatograms at 520 nm of the anthocyanins/anthocyanidins of Vaccinium Concentrate (VC).

FIG. 2B is a graph of total ion chromatograms with diode array detection of Vaccinium Concentrate (VC).

FIG. 2C is a table which depicts results from total ion chromatography/tandem mass spectrometry of major components (i.e. anthocyanins) of Vaccinium Concentrate (VC).

FIG. 3 is a chart of major phytochemicals detected by reverse phase high-performance liquid chromatograms at appropriate wavelengths coupled to mass spectrometry of Rosa Concentrate (RC) and an extract of Rosa canina L.

FIG. 4 is a composite graph of reverse phase high-performance liquid chromatography coupled to tandem mass spectrometry of catechins of Rosa Concentrate (RC).

FIG. 5 provides non-limiting examples of preferred antioxidant (anti-aging) formulations in accordance with this invention.

FIG. 6 provides non-limiting examples of preferred anti-inflammatory formulations in accordance with this invention.

FIG. 7 provides non-limiting examples of preferred wound healing formulations in accordance with this invention.

FIG. 8 is a chart which summarizes the respective importance of each ingredient in the various formulations.

FIG. 9 is a chart which depicts ORAC 5.0 analysis of Vaccinium Concentrate (VC), Rosa

Concentrate (RC), and various fruits.

FIG. 10 is a table which depicts the Cellular Antioxidant Activity (CAA) of Vaccinium Concentrate (VC) and various fruits compared to quercetin.

FIG. 11 is a table which depicts the Cellular Viability Activity (CVA) of Vaccinium Concentrate (VC) and various fruits.

FIG. 12 is a chart which depicts the effect of Vaccinium Concentrate (VC) and astaxanthin on inflammatory, growth, and adhesion factors in human umbilical vein endothelial cells (HUVECs).

FIG. 13 is a graph which depicts the trend of platelet values measured in a patient's complete blood counts (CBC) between the dates of Dec. 22, 2021 and Jun. 26, 2022. Transfusion administrations are indicated by open circles; the first (L-R) star indicates when VC was introduced into the patient's diet and the second star indicates the date of administration with dose adjustment.

### DETAILED DESCRIPTION

### Phytochemicals

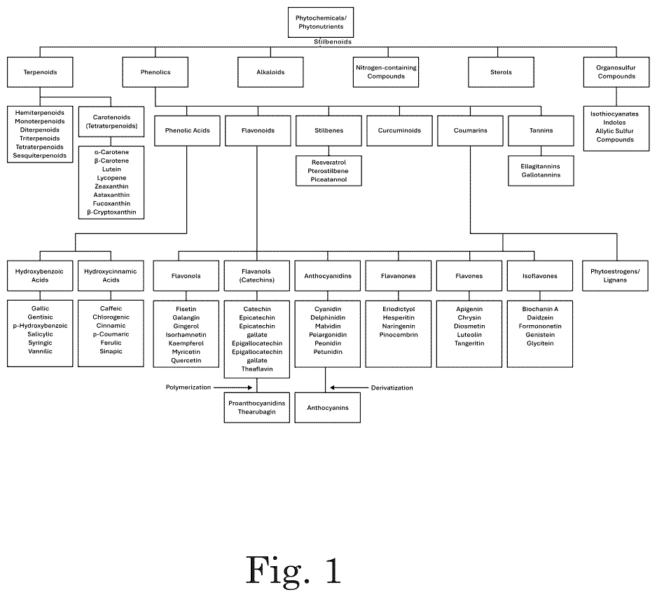

Phytochemicals are secondary plant metabolites found in vegetables, fruits, cereal grains, herbs, spices, and plant-based beverages such as tea and wine, with value as dietary supplements and in medicine. Thousands of phytochemicals have been identified but only certain ones, segregated into structural classes, have been attributed bioactivity and health benefits in mammals. See FIG. 1 for a diagram of phytochemical classes.

The presence and concentrations of phytochemicals vary widely among different plant species. Some plants may be rich in certain phytochemicals while lacking or missing others; certain families of plants are known to be rich sources of specific secondary metabolites (such as isoflavones in Fabaceae or organosulfur compounds in Alliaceae). Therefore, the phytochemical composition of a plant is unique and contributes to its specific health promoting value.

A fundamental property of phytochemicals is the ability to scavenge reactive oxygen species (ROS) to protect macromolecules from damage caused by environmental toxins and natural metabolic processes (https://www.uclahealth.org/news/what-are-phytochemicals-and-why-should-you-eat-more-them, May 10, 2023). Many (if not most) of the health benefits of phytochemicals are linked to their antioxidant activity.

Phytochemicals help to achieve and maintain balanced immune function - neither overactive nor underactive-through many mechanisms associated with antioxidant capacity. Anti-inflammatory activity reduces ongoing inflammation linked to chronic diseases. Phytochemicals may work as antimicrobial agents to reduce the chance that bacteria, fungi, parasites, or viruses establish an infection in a mammal. With ongoing infection, phytochemicals promote an appropriate immune response and facilitate wound healing. Another role for phytochemicals is improvement of endothelial function and increased vascular blood flow.

When coupled with the ability to inhibit lipid oxidation, demonstrate lipid-lowering effects, exert hypoglycemic and insulin-regulatory effects, phytochemicals have obvious benefits for cardiovascular conditions and diabetes and, tangentially, to malignancy. The antioxidant activity of phytochemicals prevents damage to DNA, and through other mechanisms, assists enzymes that repair mutated genes, promote apoptosis (programmed cell death), and regulate the cell division cycle to slow or prevent cancer growth. Research supports using phytochemicals as chemopreventive agents for several types of cancer in humans and, to a lesser extent, in companion animals.

Finally, phytochemicals also influence many claims of neurological function, such as brain plasticity, cognition, prevention or delay of neurodegenerative disorders (e.g. Alzheimer's and Parkinson's diseases) and reduction of anxiety and insomnia. For humans and companion animals, the support of phytochemicals helps with mood and the ability to adapt to sensory stimulation, to adopt new information, adjust to injury and traumatic experiences, and improve claims of cognition such as memory, attention, and learning ability.

The health effects attributed to phytochemicals are primarily due to the synergistic actions of bioactive dietary components which include micronutrients and phytochemicals. It is largely accepted that combinations of various phytochemicals in whole plant-based matrices have potentially stronger, and thereby more consistent actions than single, isolated phytochemical compounds. Consumption of a wide variety of whole plant matrices, especially in the context of foods and dietary supplements, ensures the likelihood that every phytochemical class and its benefits are available.

However, specific interactions between different phytochemicals and whether they have inhibitory effects on each other is a complex topic that likely depends on the specific phytochemicals in question, their concentrations, and the biological context. Despite the attractiveness of bioactive phytochemicals and combinations as dietary supplements or at pharmacologic doses, the possible efficacy and safety of each requires case-specific confirmation. Extremely high degrees of effectiveness are achieved by incorporating a uniquely small number of plant-based components, as low as four, as the core matrix of a supplement formulation in accordance with the present invention.

### Phytochemical Composition of Source Plants

The plants used for the core matrix of the supplement formulations of the present invention are Vaccinium, Rosa, Taraxacum, and Achillea, sourced from high latitude habitats. The reasons for choosing these species from this location are based on environmental conditions that drive their exceptional, but underexplored, phytochemical properties. Although many were considered, the four selected are replete with bioactive phytochemicals and complementary health benefits.

The plant components are sourced from high latitude environments, preferably in Alaska, in habitats where they are native or introduced and grow sustainably without cultivation. The environment of Alaska is one of the most stressful climates worldwide. The Alaska landscape is characterized by a short growing season with extended photoperiods during the summer (reaching 24 h above the Arctic Circle) and complete absence of sunlight in the winter, with vast shifts in temperature from as low as −60° C. in the winter and up to 27° C. in summer months (https://alaskapublic.org/2017/06/20/ask-a-climatologist-summer-solstice/). The intermingling of permafrost/semi-permafrost soil with highly divergent compositions and mineral contents are reflective of every major geological period. These extremes significantly stress indigenous plants to prompt adaptive biochemical responses and significantly modulate the phytochemical profiles. Biotic and abiotic stresses in the wild are endured by plants through defensive chemical adaptations, fostering typically more complex phytochemical composition in wild species compared to cultivated varieties. Wild plants tend to accumulate more concentrated phenolic compounds than their cultivated relatives, as the latter are more buffered from environmental insults due to commercial agricultural practices in temperate growing regions and have attenuated secondary phytochemical metabolism due to selection and breeding for enhanced growth and palatability. Similarly, wild plants depend on volatile or astringent compounds such as tannins, catechins, saponins, terpenoids, and tartaric acid for protection from mammal/bird/insect herbivory and microbial infections, while cultivated plants are protected from herbivory by pesticides. To suppress the growth of unwanted plants (weeds) in their vicinity, wild plants make various molecules, called allelochemicals, as natural herbicides. These molecules are either broadly distributed in the plant kingdom or species specific, as needed; examples of the former include cinnamic acid, catechins, and coumarins, and the latter, alkaloids.

These plants are integral dietary resources and traditional medicines used both orally and topically by the indigenous peoples of Alaska, Canada and Russian Far East. Most of the uses described are related to health challenges of daily living, such as skin and eye protection from the elements, cuts and scrapes, serious wounds, infections, gynecological and obstetric concerns, and pain management. Notably absent are mentions of modern health concerns of adequate antioxidant intake, immune responsiveness, weight management, reduction of chronic inflammation and prevention of chronic conditions including cardiovascular disease, diabetes, and cancer. Despite typically high body mass index (>25), modern health disorders were virtually non-existent at the time of Western contact until the 1960s. Now, serious chronic diseases are common amongst Alaska Natives due to abandonment of their traditional foodstuffs (Whitney, E and Rolfes, S R. Understanding Nutrition. South Melbourne, Victoria: Wadsworth-Thomson Learning. 2013, p. 65). Many reports document types and uses of plants for diet and traditional healing purposes, but a dearth of information exists for linking these to underlying phytochemical composition that correlate with health benefits. These observations led the present inventor to more detailed investigations of the phytochemical profiles, and applications to current health concerns described in this application.

‘context’ is an issue with phytochemicals that precludes the ability to predict accurately efficacy and safety. Examples of context include:
Source and preparation-particular species of a plant and the way it is grown, harvested, and processed can influence the concentration and activity of its phytochemicals, as described in detail in this application.
Biological variability-effects may vary depending on the consuming individual's age, genetics, health status, and even gut microbiota.
Dosage and concentration-the same phytochemical can be beneficial at one dose and harmful at another. For example, excessive consumption of certain alkaloids can be toxic.
Multiple effects-the same phytochemical may interact with multiple molecular targets; in any condition or disease state, activity may range from inhibitory to activating.
Combination effects-phytochemicals often work in synergy with other compounds and isolating them might alter efficacy or safety.
Drug interactions-phytochemicals can interact with each other or prescription medications, either enhancing or reducing their effects.

In complex phytochemical formulas as presented here, empirical evidence is required to ensure anticipated performance.

Plant Selection. As background for this endeavor, the present inventor made visits to Alaska Native villages throughout the state from 2005-2020. Interviews with elders and healers were conducted to verify published ethnobotanical disclosures from respective villages or regions. A database was compiled of these results and correlated with botanical entries for all accepted Alaska plant species according to the United States Department of Agriculture Plants Database (USDA, NRCS. 2023. The PLANTS Database (http://plants.usda.gov, Oct. 29, 2023). National Plant Data Team, Greensboro, NC USA). At the beginning of this step, approximately 5500 species and subordinate taxa from Alaska (Southeast (SE), Southcentral (SC), Southwestern (SW), Interior (IN), Far North (FN), and Aleutian (AL) regions) and overlapping Canada, namely 4,910 from British Columbia (BC) and 2,113 from the Yukon Territory (YT), were considered for further evaluation; at the conclusion, 2,947 were found to occur in Alaska with some common to the other locations. An unexpected 83% (2,446) of these from Alaska were found to be of known or possible ethnobotanical value, based on plant family or related taxa; 67% (1,974) serve as dietary resources to humans, large and small mammals, and birds. A smaller 34% (1,002) were documented to have known or potential medicinal applications. Approximately 1% (31) were flagged as toxic. Traditionally, untoward effects were ameliorated by correct use of a plant part at specified time of harvest and in appropriate amount; in the case of non-food (e.g. seasoning, tea, tonic, medicinal) plants, this was achieved by avoiding excessive consumption.

At this point, 1000-2000 plant candidates for commercial applications as dietary supplements or therapeutics were pared according to preeminence of human and companion animal use, abundance/sustainability, access/logistics, seasonality, organoleptic properties and, ultimately, phytochemical profiles. Guided by the USDA Plants Database, rare, endangered, and wetland species were eliminated from further consideration. Experience dictated that only those accessible by the limited road system and air or marine ports would be practical to pursue. Seasonality concerns related not only to logistics, i.e. avoid winter, but to a staggered calendar for optimal time of harvest for each plant in different, remote locations. Post-harvest handling possibilities (e.g. freezing) were needed to preserve overall freshness and safety that affect taste, color, aroma, and phytochemical stability prior to processing.

Candidate plants in the penultimate round of evaluation were preeminent species and subordinate taxa used as foods, seasonings, teas, tonics, and medicinals (oral and topical). The selected fruits (Vaccinium, Empetrum, Rosa, Rubus, Viburnum, Arctostaphylos, Cornus); herbs, including greens or flowers/inflorescences (Achillea, Matricaria, Artemisia, Chamaeron, Viola, Geranium, Trifolium); herbs including greens, roots, barks or catkins/tips (Taraxacum, Allium, Ligusticum, Polygonum, Oplopanax, Betula, Picea, Rhodiola, Alnus). All were harvested according to ethnobotanical experience, processed by RWD into concentrates, and evaluated for organoleptic properties.

Five, including Vaccinium, Rosa, Taraxacum, Achillea, and Betula, were selected to undergo phytochemical fingerprinting by either High-Performance Thin-Layer Chromatography (HPTLC) or High-Performance Liquid Chromatography (HPLC). Concentrates of the first four provide the core matrix of the present invention.

Because Betula concentrate proved interesting upon this initial phytochemical analysis, but impractical to process by RWD methods described here and of sporadic or low abundance for phytochemical bioactives of interest, it was pursued for use through an alternative method of preparation or source in standardized extract and purified forms.

The core matrix concentrates are prepared with a maximum number of plant organs to capture valuable phytochemical bioactives that preferentially localize in aerial parts or roots, or at specified times in the seasonal cycle such as emergence, flowering, post-flowering, or ripening. As a result, some are common among the four, others are structural variations, and certain ones are unique. Most importantly, these four are complementary and sufficiently complex to deliver every known class of phytochemicals except organosulfur compounds, by design. (Organosulfur compounds, primarily from the Alliaceae and Brassicaceae, were intentionally omitted from inclusion among the phytochemicals of the core matrix. Many of these cause intolerance reactions in humans and are debated for appropriateness or acceptable safety in the diets of dogs and horses.) Furthermore, the potency of each phytochemical is adequate to realize their associated benefits without further manipulations for dietary supplements; the matrix provided by each concentrate supports addition of any desired phytochemical bioactive for specific purposes.

Harvest and Processing. Vaccinium Concentrate (VC) is comprised of at least two preferred species, one is introgressed Vaccinium ovalifolium Sm. x Vaccinium alaskaense How. (SE, SC, AL and BC harvest sites), and the other is Vaccinium uliginosum L. (IN and YT harvest sites). V. ovalifolium was the focus of the present inventor's patent for “Vaccinium Species Compositions” (U.S. Pat. No. 8,591,964 B2) in which V. ovalifolium was hand-segregated from naturally occurring V. ovalifolium x V. alaskaense. The principal findings were exceptional amounts and complexity of the phenolic phytochemicals, anthocyanins and proanthocyanidins. The first step to maximizing anthocyanin content is harvesting when fruit is very ripe and the pericarp is nearly as purple as the skin, with red or purple leaves devoid of chlorophyll. In blueberry fruits, a developmentally coordinated shift occurs from cyanidin-type, di-substituted anthocyanins toward delphinidin-based, tri-substituted pigments during ripening (Spinardi, A et al. 2019. Front. Plant Sci. 10, 1. doi:10.3389/fpls.2019.01045). The late-harvest fruit/leaves are subjected to at least one freeze-thaw cycle, as suggested in U.S. Pat. No. 8,591,964 B2, and, since then, recognized by others for improved product quality (https://www.sciencedaily.com/releases/2014/07/140722124810.htm).

Ingredients come in a wide variety of forms. Although any may be used here, forms with greater concentration or purity, bioavailability, or extended release are preferred to accompany the core matrix that is produced by RWD with minimal processing time, heat, and oxidative exposure.

The core matrix concentrates are best used in capsule or powder supplement forms. Other forms that require more heat (gummies or sterilized drinks), pressure (tablets and lozenges), grinding or milling (fine powders), and the like may be produced, but are less appropriate due to lability of certain bioactives. Aqueous, ethanolic, aqueous ethanolic, or buffered solutions may be used to make extracts or tinctures and preparation is facilitated by the concentrated nature of the core matrix as starting material. The extracts or tincture may be used in various oral or topical preparations that must be free of any insoluble matter.

### Example 1—Preparation of VC Component of the Core Matrix

As a non-limiting example of how to prepare the VC component of the core matrix, a blend of fruit from V. ovalifolium x V. alaskaense is 75-95% by weight and V. uliginosum is 5-25% by weight; is combined at 85-95% by weight with leaves at 5-15% by weight. Appropriately handled starting materials are prepared by gentle processing to retain high levels of phytochemical bioactives in the berry concentrate. Also, as taught in U.S. Pat. No. 8,591,964 B2, RWD is employed for that purpose. This drying technology prevents degradation of even the most labile chemical structures and functions. As taught in that patent, and consistent with current industry-wide practices, frozen raw plant material is thawed to ambient temperature and disintegrated into a puree of appropriate thickness to apply physically to the dryer belt. The puree is loaded into an in-stream vat, in a quantity to completely fill. During this time, often many hours, the puree is maintained in a dispersed (not settled) state by a rotational paddle and held there during application to the belt. This procedure has been found to accelerate oxidation and degradation of labile phytochemical bioactives, such as certain phenolics.

Raw material processing prior to drying is upgraded now substantially to improve finished product quality. The preferred procedure of this invention is to 1) thaw raw material to icy slush, 2) puree raw material as icy slush using an appropriate disintegrator screen, 3) fill vat to 25-30% of capacity with icy slush, with paddling to prevent settling, while concomitantly applying to the dryer belt and, 4) continue with maintenance of 25-30% of vat capacity until processing of the whole lot of slushy material is complete. The time puree is held in the vat is further minimized by running the variable dryer belt speed at maximum rate of 1-2-minute transit time through the evaporation compartment, as opposed to 3-5-minute transit time, while achieving a final moisture content of <4%. The flakes that emerge from the dryer belt are milled to 40-60 mesh powder, optimal for functional properties and incorporation into many supplement formats.

Organoleptic evaluation describes VC as a very dark purple, shiny, free flowing powder. The product has an intense, characteristic odor for blueberries, a somewhat astringent taste with B-vitamin overtones, and a powdery to slightly grainy texture. Microbial counts, including total aerobic plate counts, yeast, molds, and various food-associated pathogens are routinely undetected or <10 colony-forming units per gram (cfu/g). This process results in a final product with a shelf-life of >5 years without noticeable change in phytochemical composition, microbial counts, flavor, aroma, or color.

### Example 2—Preparation of RC Component of the Core Matrix

As a non-limiting example of how to prepare the RC component of the core matrix, Rosa Concentrate (RC) is comprised of components from at least two preferred and closely related subspecies; >70% by weight Rosa acicularis Lindl. (prickly rose), Rosa acicularis Lindl. ssp. acicularis, Rosa acicularis Lindl. ssp. sayi (Schwein.) W. H. Lewis (SC and IN harvest sites), and <30% by weight Rosa nutkana C. Presl (Nootka rose), Rosa nutkana C. Presl var. nutkana (SC and SE harvest sites). Two naturally associated species, Rosa Woodsii Lindl (Wood's rose) and Rosa rugosa Thunb. (rugose rose) are included in RC at <5% by weight (SE harvest sites). The preferred time of harvest is September-October, when the hypanthium is uniformly ripe; in certain microhabitats, Rosa may appear ripe by June but harvesting with early ripening is avoided due to aberrant, undesirable phytochemical composition of the hip.

Dehydrated, whole rosehip material for RC is prepared by RWD as described for VC in Example 1. RC includes all parts of the rosehip, including the hypanthium (outer pulp layer), achene with seeds, peduncle, and sepals. The parts are pureed together as icy slush prior to drying to produce uniformity of the finished product. Maintaining low temperature and continuous, small batch processing with minimal stirring of settling materials, prior to application on the dryer, as described for VC, is required to minimize thermal and oxidative exposure of exceptionally valuable, but labile phytochemical bioactives. Especially susceptible are important antioxidants, such as Vitamin C, and sensitive seed oils. The time puree is held in the vat is further minimized by running the variable dryer belt speed at maximum rate of 1-2-minute transit time through the evaporation compartment, as opposed to 3-5-minute transit time, while achieving a final moisture content of 1-2%. The moisture content of the dried material is routinely <1% to undetectable by standard methods to measure water activity or loss on drying. Like VC, the dehydrated flakes are milled to 40-60 mesh powder, optimal for functional properties and incorporation into many supplement formats.

Organoleptic evaluation describes RC as a light brownish-orange, free flowing powder. The product has a light, spice odor and flavor with a fine, grainy texture. Microbial counts, including total aerobic plate counts, yeast, molds, and various food-associated pathogens are routinely undetected or <10 colony-forming units per gram (cfu/g). This process results in a final product with a shelf-life of >5 years without noticeable change in phytochemical composition, flavor, aroma, or color.

### Example 3—Preparation of TC and AC Components of the Core Matrix

As a non-limiting example of how to prepare the TC component of the core matrix, Taraxacum Concentrate (TC) is comprised of one preferred species and closely related subspecies, identified by botanical experts as Taraxacum officinale F.H. Wigg., Taraxacum officinale F.H. Wigg. spp. ceratophorum (Ledeb.) Schinz ex Thell., and Taraxacum officinale F.H. Wigg. ssp. officinale that comprise ˜90% by weight (principally SC, SE, SW, and IN although may be harvested in any region). Lower abundance, lesser-known species in the composition and may include Taraxacum carneocoloratum A. Nelson, Taraxacum eriophorum Rydb., Taraxacum laevigatum (Willd.) DC., Taraxacum lyratum (Ledeb.) DC., and Taraxacum phymatocarpum J. Vahl that comprise ˜10% by weight of the TC component (may be harvested in any region). Taraxacum is harvested while in flower (not seed), to capture the unique phytochemical composition associated with that plant part and phase of growth. The preferred time of harvest is late-May through June, at the vigorous emergence phase of growth; thereafter, growth is only sporadic throughout the remainder of summer, unlike observed for Taraxacum growing at more temperate latitudes. A standard analysis based on high performance thin layer chromatography (HPTLC) clearly placed plant material harvested for use in this work in the genus Taraxacum. Although very similar to T. officinale, assignment at the species level was not possible. Regardless of plant part (i.e. leaf or root/taproot) analyzed, both were considered phytochemically similar but from a novel species.

As a non-limiting example of how to prepare the AC component of the core matrix, Achillea Concentrate (AC) is a complex consisting of preferred Achillea species including Achillea millefolium L. (common yarrow), and intermixed subspecies Achillea millefolium L. var. alpicola (Rydb.) Garrett (common yarrow), Achillea millefolium L. var. borealis (Bong.) Farw. (boreal yarrow), Achillea millefolium L. var. nigrescens E. Mey. (common yarrow), Achillea millefolium L. var. occidentalis DC. (western yarrow), and Achillea millefolium L. var. pacifica (Rydb.) G. N. Jones (Pacific yarrow), at ˜95% by weight (principally SC, SW, AL, IN, although may be harvested in all regions) with Achillea ptarmica L. (sneezeweed), and Achillea sibirica Ledeb. (Siberian yarrow), together ˜5% by weight of final composition (SC, SW, IN although may be harvested in all regions). Achillea is harvested only while in flower, to capture the most desirable phytochemical composition associated with that plant part. The preferred time of harvest is mid-August to mid-September.

Similarly to VC and RC, TC and AC are gently dried with minimal heating, preferably processed with RWD. For TC, the aerial parts and root/taproot are separated at harvest and the root/taproot are sprayed with clean, cool water to carefully remove soil, sand, and residual material. This step is important to ensure uniform pureeing and to prevent damage to the disintegrator unit. Cleaned plant materials are weighed to achieve desired ratios of aerial parts to root/taproot in 22-pound (or 10-kilogram) portions, placed together in food grade plastic bags, and frozen until processing. The procedure is similar to that for VC and RC and entails 1) thaw frozen raw material to icy slush, 2) puree raw material as icy slush using an appropriate disintegrator screen, 3) fill vat to 25-30% of capacity with icy slush while paddling to prevent settling with concomitant application to dryer belt and, 4) continue with maintenance of 25-30% of vat capacity until processing of the whole lot of slushy material is complete. The time puree is held in the vat is further minimized by running the variable dryer belt speed at maximum rate of 1-2 minute transit time through the evaporation compartment, as opposed to 3-5 minute transit time, while achieving a final moisture content of 1-2%. The flakes that emerge from the dryer belt are milled to 40-60 mesh powder. The ultimate TC is a dehydrated product of 50-80% by weight aerial parts and 20-50% by weight root/taproot; the dominance of one part versus the other addresses different therapeutic purposes.

AC is processed with frozen, aerial parts for 100% by weight but, unlike TC, does not include roots. The moisture content of the dried material is routinely <1% to undetectable by standard methods to measure water activity or loss on drying. The dehydrated flakes are milled to 40-60 mesh powder, optimal for functional properties and incorporation into many supplement formats. Especially susceptible to loss on drying are important antioxidants and volatiles in the essential oil, such as mono-and diterpenes.

Organoleptic evaluation describes TC as greenish-light brown and AC as greenish-light gray with white flecks, free flowing powders. Both concentrates have herbal but distinct odors, characteristic of retained volatiles, and comparable bitter, slight herbal flavors with light, powdery textures. Microbial counts, including total aerobic plate counts, yeast, molds, and various food-associated pathogens are routinely undetected or <10 colony-forming units per gram (cfu/g). This process results in a final product with a shelf-life of >5 years without noticeable change in phytochemical composition, flavor, aroma, or color.

General Phytochemical Observations. Initial phytochemical fingerprinting by the present inventor established basic features of each concentrate in the core matrix. VC was confirmed to be the principal source of phenolics in its phytochemical composition. The species of Vaccinium that can grow in the environment of Alaska with uninterrupted UV exposure of long summer photoperiods exclusively contribute abundant anthocyanins, a subclass of phenolics. Anthocyanins appear to be segregated by different sections of the Vaccinium genus. V. ovalifolium and V. alaskaense are classified in Sect. Myrtillus, whereas V. uliginosum is classified alone as the type species in Sect. Vaccinium. Other phenolics include phenolic acids, flavonoids, particularly flavonols, flavones and flavanols, stilbenes, tannins, proanthocyanidins, phytoestrogens/lignans and coumarins (https://www.frontiersin.org/research-topics/56564/the-nutritional-and-health-benefits-of-vaccinium-berries). Flavanones presumably are rare and isoflavonoids have not been detected in Vaccinium species.

Rosa Concentrate (RC) is the other principal contributor of phenolic acids to the core matrix and contains replete beneficial phytochemicals such as an array of hydroxycinnamic phenolic acids, flavonoids such as flavonols, catechins, anthraquinones, saponins, terpenoids, carotenoids, fatty acids, tannins, aldehydes, phenolic/alcoholic compounds, minerals, and high levels of Vitamin C (Verma, A et al. 2020. Future J. Pharm. Sci. 6, 114. doi: 10.1186/s43094-020-00132-z). RC contains low and trace amounts of flavanones and isoflavones. Rosa is notoriously unpredictable and highly variable with respect to growth characteristics; this also holds true for lability of certain nutrients and phytochemical bioactives, e.g. Vitamin C, carotenoids, quercetin, catechins, and ellagic acid, as well as phytochemical content that displays pronounced variation in different species of Rosa (Winther, K et al. 2016. Botanics: Targets and Therapy, 6, 11. doi: 10.2147/BTAT. S 91385). The composition of RC, although comparable to that of R. canina L. and other Rosa species, is stable; most likely, this is the result of combining four species, harvested within a tight time window, and processed very gently by RWD, into one final RC concentrate to mitigate dramatic lot-to-lot variation in the composition of a typical commercial product comprised of a single species.

Taraxacum Concentrate (TC) and Achillea Concentrate (AC) are included in the formulation to expand representation of bioactive phytochemicals characteristic of VC and RC. TC and AC are the principal contributors of terpenoids from various subclasses including mono-, di-, tri-, sesquiterpenes and a plethora of derivatives of each. Other major phytochemical classes of Taraxacum are pentacyclic triterpenoids, sesquiterpene lactones, carotenoids (technically known as tetraterpenes), flavonoids and phenolic acids, polysaccharides, and

sterols (Duan, L et al. Molecules. 2020, 25, 3260. doi:10.3390/molecules25143260). Achillea is known also for sesquiterpene lactones, flavonoids, coumarins, alkaloids, alkanes, saponins, polyacetylenes, sterols, guaianolides, and essential oils (Barda, C et al. Sci. Pharm. 2021, 89, 50. doi:10.3390/scipharm89040050). The Taraxacum and Achillea species growing in Alaska are considered by botanists to be cosmopolitan, distributed all over the globe, except in the most extreme high Arctic and Antarctic. They are highly adaptable to a range of climatic and environmental conditions, unlike the Vaccinium and Rosa species that are native and restricted (although not considered endemic) to Alaska.

Curcumin was not included in any formula described herein despite being a recognized antioxidant and anti-inflammatory. Many humans take it already and it is less-well studied in horses and dogs that may experience digestive issues, allergic reactions, bleeding and find many forms of it unpalatable. Astaxanthin is not used for horses because it is very expensive to use in these combination formulas, doesn't really add much, and is not particularly palatable to them. Apple peel is not used for dogs for pretty much the same reasons. Humans, with an omnivore diet and nutritional needs get both.

Detailed Chemical Analysis of VC, RC, TC, and AC. As a final step to suggest potential uses of the core matrix in dietary supplements and therapeutics with multiple indications, more powerful analyses were employed by the inventor to elaborate phytochemical compositions of VC, RC, TC, and AC. The initial phytochemical fingerprints obtained from HPTLC or reverse phase HPLC alone revealed rich fingerprints of Vaccinium and Rosa (to a lesser extent) that confirmed distinct differences from species that grow in more temperate latitudes and are unable to survive in Alaska. Conversely, high similarity was observed for analyses of Alaska species compared to reference analyses from Taraxacum and Achillea species growing at lower latitudes. Non-volatile phytochemicals were evaluated by appropriate conditions widely reported for reverse phase HPLC coupled with mass spectrometry (HPLC-MS) or tandem mass spectrometry (HPLC-MS/MS), or total ion chromatography (TIC) coupled with tandem mass spectrometry (TIC-MS/MS). These instruments were equipped with UV-VIS diode array detectors which are very useful in broadly identifying compounds. Gas chromatography coupled with tandem mass spectrometry (GC-MS/MS) was added for analyses of volatiles in TC and AC.

Vaccinium. Evaluation of VC was conducted first, and in most detail, due to the relative prominence of phenolics in this dietary plant. Total phenolics, although abundant in Vaccinium, are notoriously variable and generalized comments reflect best-estimates. Phenolics in commercial Vaccinium species range from 174 to 756 mg/100 g fresh weight, or approximately 17 to 76 mg/g dry weight without accounting for losses from processing (https://www.frontiersin.org/research-topics/56564/the-nutritional-and-health-benefits-of-vaccinium-berries). Phenolics in VC consistently average 85 to 95 mg/g dry weight after processing; of this total, 40-50 mg are comprised of anthocyanidins/anthocyanins.

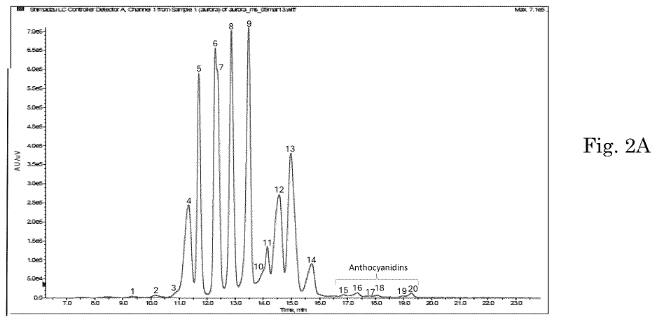

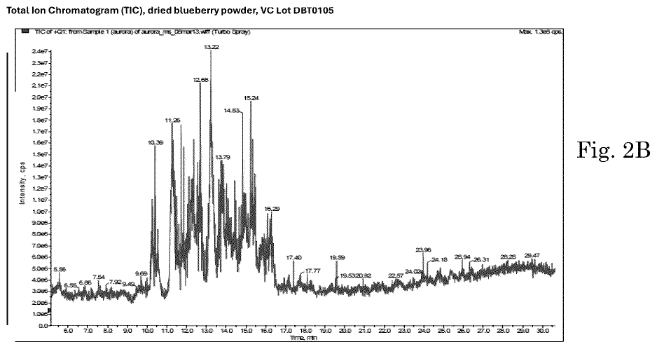

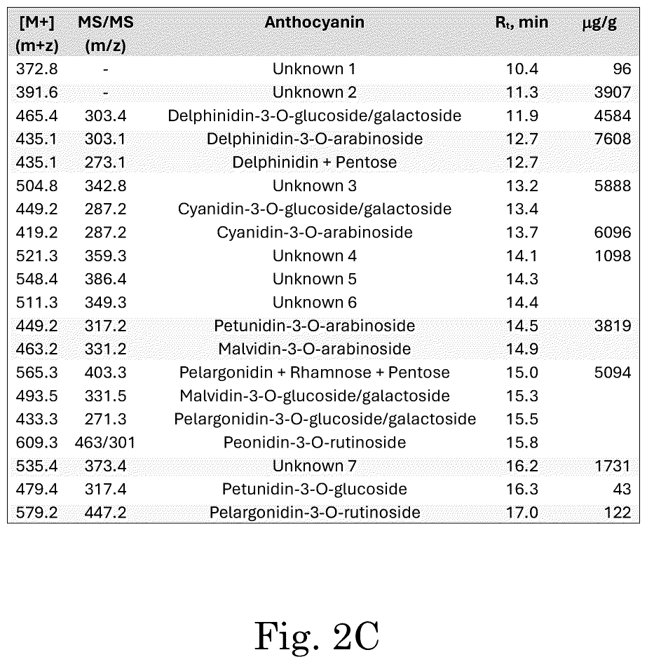

Analysis by standard reverse phase HPLC fingerprint reveals at least 14 prominent anthocyanins, with more at lower abundance, based on five anthocyanidin cores, delphinidin, cyanidin, malvidin, peonidin, and petunidin (FIG. 2A). A previous analysis of a similar VC sample, disclosed in U.S. Pat. No. 8,591,964 B2, identified three other anthocyanins. Subsequent TIC coupled with MS/MS identified more anthocyanins in the VC sample (FIG. 2B) amounts of cyanidin, delphinidin, and derivatives (mostly glucosides and galactosides) of both, with delphinidin mostly contributed by V. ovalifolium x V. alaskanse, followed by glycosylated derivatives of malvidin, peonidin, petunidin, and pelargonidin. V. uliginosum contains predominantly cyanidin and cyanidin glycosides in its composition, along with considerably more malvidins than the other species, as well as an appreciable amount of Vitamin C. V. ovalifolium x V. alaskanse and V. uliginosum possess plentiful anthocyanins and proanthocyanidins, but with respective profiles, consistent with classification in different taxonomic sections of the Vaccinium genus. Of these, V. ovalifolium and V. uliginosum were studied after the filing of U.S. Pat. No. 8,591,964 B2 (Kellogg, J et al. J. Agric. Food Chem. 2010, 58, 3884. doi:10.1021/jf902693r). The reported results are in overall agreement regarding VC anthocyanins, except without identification of pelargonidin, and proanthocyanidins; qualitatively and quantitatively, V. ovalifolium and V. uliginosum profiles reported in this article are not as complex or abundant as presented for VC.

Commercial Vaccinium species typically produce five of the six major, core anthocyanidins that possess different derivatization patterns, particularly glycosylations (Petruskevicius, A. et al. Horticulturae. 2023, 9, 288. doi:10.3390/horticulturae9020288). The total anthocyanin content of Vaccinium fruits, represented by V. angustifolium, V. corymbosum, and V. myrtillus typically ranges from 100 to 700 mg/100 g fresh weight (Kalt, W et al. Adv. Nutr. 2020, 11, 224. doi:10.1093/advances/nmz065; Mallik, AU and Hamilton, J. J. Food Sci.

Technol. 2017, 54, 1545. doi:10.1007/s13197-017-2586-8; Petruskevicius A et al. Horticulturae 2023, 9, 288. doi:10.3390/horticulturae9020288; Gizzi, C et al. Eur. Rev. Med. Pharmacol. Sci. 2016, 20, 2418). The anthocyanin content of V. membranaceum was found to be 100-170 mg/100 g fresh weight and V. ovatum between 560-625 mg/100 g fresh weight (Lee, J et al. J. Agric. Food Chem. 2004, 52, 7039. doi:10.1021/jf049108e).

These results indicate that V. myrtillus (from Northern Finland) has the highest anthocyanin content among the Vaccinium species examined, followed by V. corymbosum and V. angustifolium; V. membranaceum has a relatively low anthocyanin content compared to other Vaccinium species. Others have reported anthocyanin content of 265 to 311 mg/100 g fresh weight for V. ovalifolium and 206 to 310 mg/100 g fresh weight for V. uliginosum from Alaska (Kellogg, J et al. J. Agric. Food Chem. 2010, 58, 7, 3884. doi:10.1021/jf902693r). From Finland, V. uliginosum anthocyanins from samples gathered along a north-south axis were 1425+/−398 mg/100 g dry weight, with greatest values corresponding to highest latitudes (Lätti, A K et al. J. Agric. Food Chem. 2010, 58, 427. doi:10.1021/jf903033m). After processing, anthocyanin content of VC is 40-50 mg/g dry weight, consistent with fresh weights at or above the high end for species from Alaska and Finland, due to post-harvest handling as described here and in U.S. Pat. No. 8,591,964 B2. Processing yields are routinely 90 to 95% of fresh weight anthocyanins, even for those most labile, with an overall concentration of 9 to 10 times total fresh weight.

Notably absent from almost all reports of Vaccinium anthocyanidins is pelargonidin, with the exception of two noncommercial species of Vaccinium from the Far East that have been shown to possess pelargonidin-based anthocyanins. Vaccinium japonicum Miq. (mountain blueberry) and Vaccinium bracteatum Thunb. (Asiatic bilberry), both of which are wild blueberry species native to Japan and China, contain them (Su, Z. Pharmaceutical Crops 2012, 3, 7. doi:10.2174/2210290601203010007; Zhang, Y-L. Front. Plant Sci. 2021, 12, 697212. doi:10.3389/fpls.2021.697212). Only the 3-O-arabinoside and 3-O-xyloside of pelargonidin have been isolated from the berries of V. japonicum, whereas V. bracteatum possesses five derivatives of pelargonidin (pelargonidin-3-O-galactoside, pelargonidin-3-O-glucoside, pelargonidin-3,5-O-diglucoside, pelargonidin-3-O-rutinoside, and pelargonidin-3-O-arabinoside). Three derivatives of pelargonidin (pelargonidin-3-O-galactoside, pelargonidin-3-O-glucoside, pelargonidin-3-O-rutinoside) were detected in VC. The exceptional natural anthocyanin content and complexity is the most notable phytochemical feature of VC, despite inexhaustive analysis, compared to other commercial Vaccinium products.

In addition to the anthocyanins identified in the TIC-MS/MS, five unknowns were identified but likely were not anthocyanins; two unknowns generated by MS-MS appear to be completely unrelated to known anthocyanins (FIG. 2C). Candidates found in a database of chemical structures are: Unknown 1, chrysoeriol 8-C-glucoside (flavone derivative) isoschaftoside (flavone derivative), Taxchinin G (diterpenoid); Unknown 2, Hederacoside C (triterpene saponin); Unknown 3, maltotriose (polysaccharide); Unknown 4, g-carotene (teraterpenoid) Unknown 5, thalsimine or (alkaloids); Unknown 6, isoschaftoside, and; Unknown 7, thalsimine or thalsimidine. Of these, underivatized chrysoeriol and maltotriose have been identified previously in leaves of Vaccinium species (Li, Z-L et al. Zhongguo Zhong Yao Za Zhi. 2008, 33, 2087; Akšić, M F et al. Plants (Basel). 2019, 8, 205. doi:10.3390/plants8070205); γ-carotene is a possibility (Hera, O et al. ACS Omega. 2023, 8, 18603. doi:10.1021/acsomega.3c00466). The others have not been reported from Vaccinium, although isoschaftoside and thalsimine were identified here in Taraxacum.

In addition to anthocyanins, VC is particularly rich in proanthocyanidins. Of note, prior analysis of V. ovalifolium for U.S. Pat. No. 8,591,964 revealed a unique proanthocyanidin composition of equal similarity to blueberry, bilberry, and cranberry. The discovery of proanthocyanidin A-type dimers and, of particular interest, trimers, was later confirmed for V. ovalifolium and V. uliginosum (Kellogg, J et al. J. Agric. Food Chem. 2010, 58, 7, 3884. doi:10.1021/jf902693r). Collectively, the attractive phenolics content of Alaska Vaccinium species prompted the suggestion of commercialization, albeit a decade after the pioneer filing of U.S. Pat. No. 8,591,964 (Kellogg J et al. J. Entrepren. 2011, 20, 77. doi:10.1177/097135571002000104).

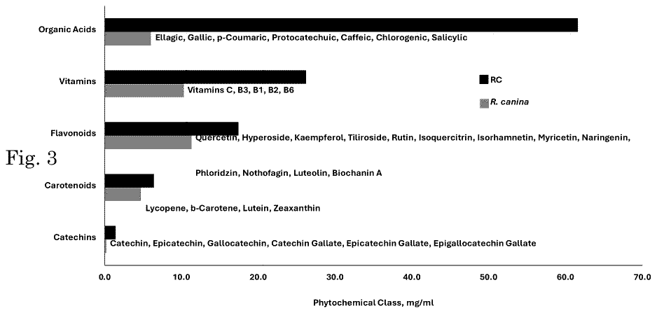

Rosa. To assess complementarity of RC to VC, extracts were evaluated by HPLC-MS, under comparable experimental conditions as extracts of VC. The main classes of phytochemicals found consistently in RC are comparable to those detected in side-by-side analyses of Rosa canina L. (FIG. 3), touted in the literature for its considerable health benefits (Winther, K. Botanics: Targets and Therapy, 2016, 6, 11. doi:10.2147/BTAT.S91385; Mármol, I et al. Int. J. Mol. Sci. 2017, 18, 1137. doi:10.3390/ijms18061137).

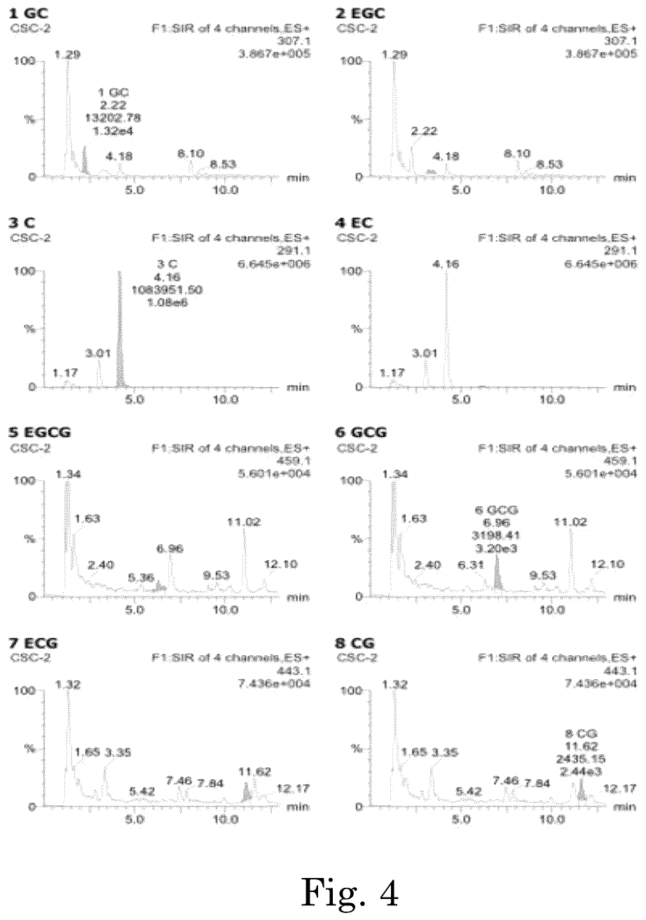

Outliers from the composite profile of Rosa species include the phenolic acids, gallic and ellagic acids, that together reach remarkably high levels between 60 to >90 mg/g dry weight. Total catechins are remarkably >1.4 mg/g dry weight, with catechin (C) consistently most abundant, followed in far lower and highly variable amounts of gallocatechin (GC), epigallocatechin (EGC), and epigallocatechin gallate (EGCG), then gallocatechin gallate (GCG), catechin gallate (CG) and epicatechin gallate (ECG), with epicatechin (EC) occurring in trace amounts (FIG. 4). The most consistently plentiful flavonoid is the flavonol, quercetin, and hyperoside (quercetin 3-O-galactoside), followed by fluctuating, interchangeable amounts of the flavonols, kaempferol or rutin, then isoquercitrin, isorhamnetin, myricetin, and naringenin (flavanone), with minor amounts of phloridzin and nothofagin (dihydrochalcones), traces of luteolin (flavone), and biochanin A (methylated isoflavone). Other compounds, characteristic of rose hips, are found sporadically in RC and include the rosmarinic acid (phenolic acid); volatiles identified by GC-MS/MS include citronellol (monoterpenoid), carvacrol and thymol (monoterpenoid phenols), and limonene (cyclic monoterpene). The red color of RC is from lycopene, detected at the highest level of 4.89 mg/g. No anthocyanins were detected and the main anthocyanidin identified is colorless leucocyanidin, in very low quantity; cyanidin and its derivatives, common in other rose hips, only occur in trace amounts in RC. Vitamin C content of RC averages a substantial 25 mg/g, consistent with between 300 mg and 1300 mg of vitamin C per 100 g fresh weight of edible hypanthium; when adjusted for concentration upon drying, and inclusion of whole hip, rather than only hypanthium, the estimated amount of Vitamin C in RC is >1300 mg/100 g fresh weight. The substantive flavonoid content of RC protects stability, promotes absorption and enhances antioxidant potency of the vitamin.

Taraxacum. The Taraxacum sourced for TC was analyzed further by GC-MS/MS for volatiles identified by organoleptic evaluation. Separate extracts of leaf, flower, and root revealed partitioned volatiles. The leaf contained a series of aldehydes including E-2-hexenal; 2-hexenal; hexanal; isovaleraldehyde; butanal, 2-methyl; benzaldehyde; hyacinthin (phenylacetaldehyde), and; 2-hexen-1-ol (fatty alcohol). In flower was 1-pentanol (alkyl alcohol); hexanal and 2-hexenal (aldehydes); 2-hexen-1-ol; β-pinene and α-pinene (terpenes); eicosane (alkane); Hyacinthin. In root was cyclopentasiloxane (organoheterosilanes); cyclopropaneoctanoic acid (fatty acid); hexadecanoic acid (saturated long-chain fatty acid), and; 3-deoxy-d-mannoic lactone. Epinephrine and, possibly, a similar type of catecholamine were detected in both roots and flowers.

Extracts of leaf, flower, and root were analyzed by HPLC DAD-MS/MS that revealed several non-volatile phytochemicals in the 100-750 m/z range. MS scans were conducted in both negative and positive modes. The major flavonoids detected were derivatives of flavones, luteolin 7-O-rutinoside in leaves and roots, apigenin glucosides in leaves, apigenin dihexoside in leaves and roots, and luteolin 7-O-glucoside in leaves, flowers, and roots. Other common flavonoids detected in flowers are underivatized luteolin and chrysoeriol as its 3′-methoxy derivative (flavones). Taraxinol, a novel phenolic, was detected in root along with several other important phytochemicals such as cis-caftaric acid were identified (alkyl caffeate esters with hydroxycinnamic acid backbone). Also detected were the taraxinic acid (sesquiterpene lactone), along with the taraxerol, taraxasterol, and taraxasterol acetate (pentacyclic triterpenoids), β-amyrin (pentacyclic triterpenol) in leaf and root, and in only root, lupeol acetate (acetylated pentacyclic triterpenoid), taraxinic acid β-D glucopyranosyl ester (sesquiterpene) and the 8-O-β-d-glucopyranoside of austricin (sesquiterpene lactone glycoside). For more qualitative analysis of some unusual putative compounds, HPLC DAD-MS/MS, tandem mass spectrometry was employed to increase the specificity and help in detecting low abundance, obscure compounds such as saikosaponin C (triterpenoid) along with trace amounts of thalsimine (alkaloid) in the root. More abundant β-sitosterol, stigmasterol, and campesterol (sterols) were found in leaf and root, and inulin (polysaccharide), in root.

Achillea. Further analysis of Achillea by HPTLC and HPLC compared to an extensively analyzed sample of A. millefolium aerial parts (i.e., leaf plus flower) revealed a surprisingly similar phytochemical footprint, but not identical, due to the inclusion of a minor amount of other Achillea species in AC for the purpose of expanding its breadth of phytochemical bioactives. Principal phenolics are the hydroxybenzoic acids, salicylic and gallic acids, and the hydroxycinnamic acids, chlorogenic and trans-cinnamic acids. These prominent phenolics consistent with those found by others on different species of Achillea include a series of caffeoylquinic acids, along with p-coumaroylquinic acid (Strzępek-Gomółka, M et al. Oxid. Med. Cell Longev. 2021, m6643827. doi:10.1155/2021/6643827). Various flavonoids include the flavonols, kaempferol, isorhamnetin and its glycosylated derivatives, isorhamnetin-3-O-glucoside and isorhamnetin-3-O-rutinoside, and hyperoside and rutin (sophorin), the glycosylated derivatives of another flavonol, quercetin; also identified were the flavanone, hesperetin, and its glycoside, hesperidin. The flavones apigenin, luteolin, and their -7-O-glucosides are visible on HPLC chromatograms as some of the highest peaks, consistent with the profile of Taraxacum, another genus in the Asteraceae family. Terpenoids include, thymol and carvacrol (monoterpene phenols); 1,8-cineole (eucalyptol) (terpene oxide); α-pinene, camphor, and borneol (bicyclic monoterpenoids with different derivatizations); azulene, chamazulene, and matricin (sesquiterpenes), and; artemisia ketone (sesquiterpene lactone) (Zanfirescu, A. Sci. Pharm. 2020, 88, 26. doi:10.3390/scipharm88020026). Guaianolides of unconfirmed identification, are from the flower, and β-sitosterol (principal sterol), and betaines (nitrogen-containing compounds) are from the leaves (Saeidnia, S et al. DARU Journal of Pharmaceutical Sciences. 2011, 19, 173). The rich phytochemical profiles identified here are not exhaustive, but contribute to the respected medicinal properties of TC and AC. Because TC and AC have rich phytochemical compositions but similar to those from lower latitudes, bioassays were not performed on these concentrates; however, the combination of TC and AC with VC and RC is novel.

Formulations. The core matrix of the present invention is a concept for phytochemical formulations with numerous health effects. The basis of the core matrix is four plant concentrates, VC, RC, TC, and AC with food and medicinal value. The particular species, region of growth, and plant organ used, satisfies inclusion of every major phytochemical class except one, bioactive organosulfur compounds, that was purposely excluded from the foundation formulation.

Each component concentrate was evaluated initially alone or in combination with others; based on uniqueness of its chemical profile and market potential, priority was given to VC, followed by RC, then TC, and AC. Birch bark containing betulinic acid and its metabolic precursor, betulin, also were evaluated for inclusion in certain formulations in accordance with this invention. In the most simplistic form, a blend of VC:RC:TC:AC (1:1:1:1) or VC:RC:TC:AC:BA (1:1:1:1:1) as a general dietary supplement, is suitable for humans, dogs, and horses.

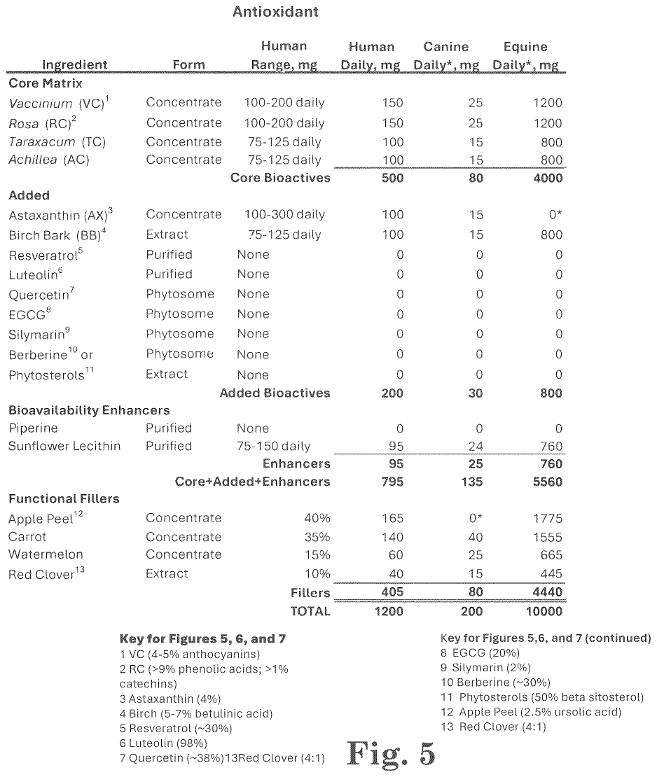

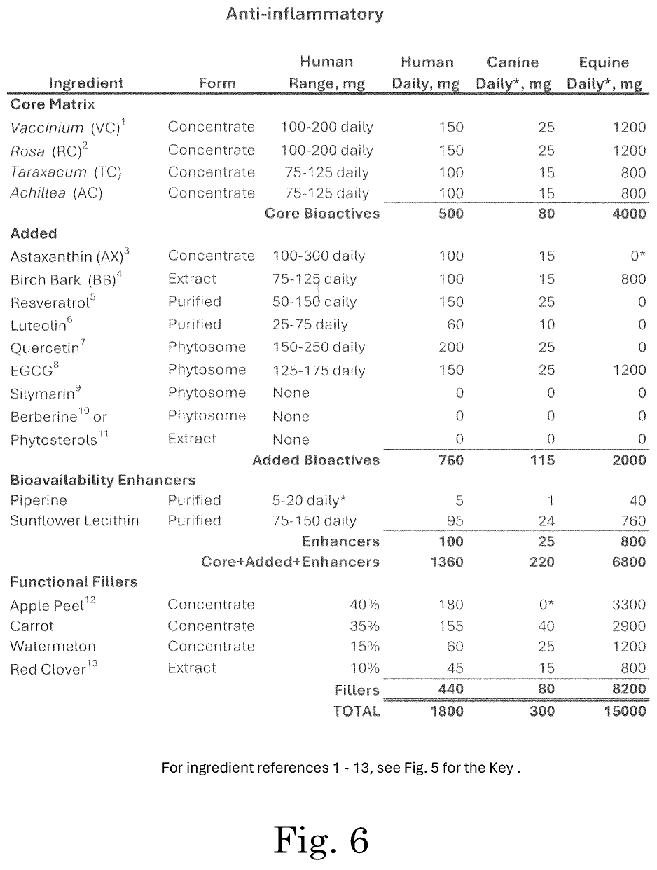

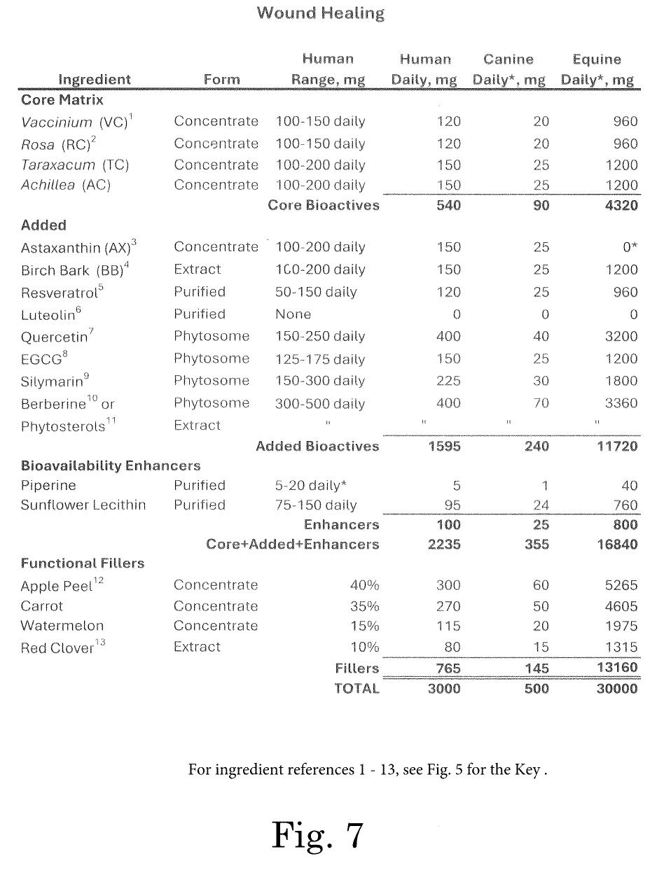

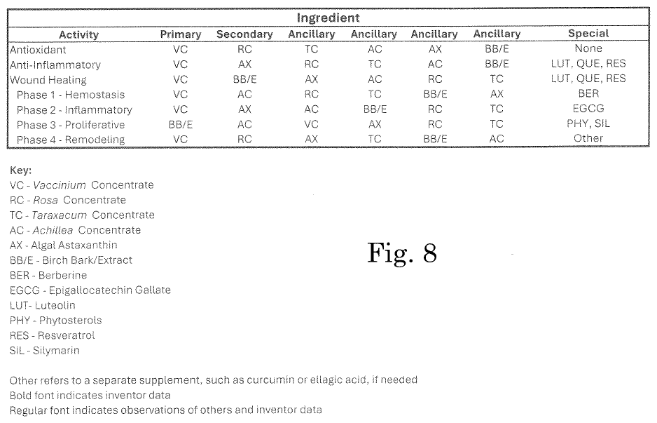

Each formulation in accordance with the present invention includes concentrates of the components of the core matrix: Vaccinium, Rosa, Taraxacum, and Achillea in amounts that reflect their phytochemistry, bioactivity, and applicability to its purpose as an antioxidant, anti-inflammatory, or wound healing product. Other active ingredients are optionally added to adapt the formulation to desired functionality. For example, astaxanthin and birch bark are included in an antioxidant version of a formulation of the present invention (FIG. 5). Astaxanthin, birch bark, plus resveratrol, luteolin, quercetin, and EGCG is included in an anti-inflammatory formulation of the present invention (FIG. 6); and astaxanthin, birch bark plus resveratrol, luteolin, quercetin, EGCG, silymarin, berberine or phytosterols are desirably included in a wound healing version of such formulations (FIG. 7). Berberine is included in a further refinement of the wound healing formula for pain associated with Phase 1 or phytosterols included in a version of the wound healing formula to enhance Phase 3; concurrent administration of a separate supplement (e.g. curcumin or ellagic acid) may further promote reconstruction and epithelial smoothness in Phase 4. Artemisinin is added to the anti-inflammatory formula for use in treatment of osteosarcoma, and related tumors; although this formulation may be used in humans, it particularly is for dog breeds at risk for this cancer. FIG. 8 is a table that summarizes the respective importance of each ingredient in the antioxidant, anti-inflammatory, and wound healing formulas.

The core matrix has a powerful antioxidant capacity, which makes it an excellent base for any condition related to chronic inflammation. In humans, these primarily include: cardiovascular diseases resulting in damage to blood vessels, contributing to conditions like atherosclerosis, heart disease, and stroke; type-2 diabetes through interference with insulin signaling, leading to insulin resistance and elevated blood sugar levels; obesity from excess fat tissue that can produce inflammatory molecules; chronic kidney disease through damage to kidney tissues, impairing their function over time; neurodegenerative diseases such as Alzheimer's disease and Parkinson's disease; asthma and allergies due to inflammation in the airways; inflammatory bowel diseases (IBD) such as Crohn's disease and ulcerative colitis in the digestive tract; autoimmune diseases like rheumatoid arthritis, lupus, and multiple sclerosis that involve the immune system attacking healthy tissues, and; cancer from DNA and other molecular damage (often linked to oxidation).

Chronic inflammation in dogs is linked to: arthritis causing joint pain and reduced mobility; inflammatory bowel disease (IBD) that leads to persistent vomiting, diarrhea, and weight loss; allergies initiated by allergens in skin (dermatitis) or ear infections (otitis); autoimmune diseases including lupus or autoimmune hemolytic anemia, and; cancer by contributing to the development of tumors. In horses, chronic inflammation may be associated with: laminitis of the hoof, often linked to metabolic disorders or obesity; equine metabolic syndrome (EMS) through insulin resistance and obesity; Cushing's Disease (PPID) characterized by a hormonal disorder causing systemic inflammation and related complications; osteoarthritis from chronic joint inflammation leading to stiffness and lameness, and; leaky gut syndrome in the digestive tract, potentially linked to poor diet or stress. Cancer does occur in horses, albeit far less frequently and of fewer types than in humans and dogs, but indicators of inflammation have been observed with the disease.

By introducing more targeted phytochemical ingredients to the core matrix, any of these conditions may be addressed with improved focus and efficacy.

The antioxidant formula (FIG. 5) is designed to be preventive for background or chronic inflammation, whereas the anti-inflammatory formula (FIG. 6) is for shorter-term conditions that arise from physical injury, irritation, or immune responses, such as sprains and strains while the body repairs damaged ligaments or muscles, sunburn, allergic reactions to insect bites, food intolerances, hives, chemical irritation, foreign body provocation, and autoimmune flare-ups. The wound healing formula (FIG. 7) addresses more serious inflammation, but not chronic, and accompanying pain and itching.

Although organosulfur phytochemicals are not included in the core matrix, the present invention contemplates formulations for specific purposes to be ingested by humans without allergies or intolerances to Brassica or Allium sources of dietary organosulfur compounds. These concentrates may be added with an adjusted ratio of VC:RC:TC:AC:X (1:1:1:1:1). Brassica or Allium sources are not preferred for dog or horse formulations, due to intolerance or toxicity. Likewise, apple peel is omitted from the dog formula whereas astaxanthin is not included for horses, mostly due to respective sensitivities or unpalatability. Curcumin is not included in any version (despite its popularity and research revealing its benefits, especially in humans) because side effects may include allergic reactions, bleeding, digestive upset, and unpalatability.

Dosages. ‘Functional fillers’ may be added to provide a desirable volume to encapsulated ingredients for humans and dogs; approximately 625 mg is adequate fill for the popular 00 capsule size. Horse formulations are administered as 10 g to 30 g top dressing, extended in volume with improved palatability. Functional fillers are whole concentrates of apple peel, carrot, watermelon, and a crude extract of red clover. Functional fillers provide their own bioactives with chemical structures that are identical (redundant), structurally related (expansive) or unique to others in the formulation that are effective against certain maladies. As principal bioactives, but not limited to, ursolic acid standardized to 2.5% by weight in apple peel concentrate; lycopene in watermelon; polyynes (polyacetylenes), falcarindol and falcarindiol, in carrot, and; mixed isoflavones in red clover extract. These components of the formulation improve texture through ingredient dispersal and enhance palatability to horses. By expanding volume, they deliver, in a top-dressing format, the added bioactives present in small quantities.

Antioxidant Dosage. The preferred daily antioxidant dose for humans is two or three capsules (based on capsule size), once or twice daily, every day for long-term use. This recommendation is based on a reference body weight of 150 pounds (˜70 kg) and may be adjusted accordingly. The preferred daily antioxidant dose for dogs is one capsule per 25 pounds (˜11 kg) of body weight, administered as once daily, dependent on breed size, every day for long-term use. The preferred daily antioxidant dose for horses is one scoop per 1000-1200 pounds of body weight (˜500-550 kg), once daily, every day for long-term use. The dose may be adjusted for draft or miniature horses, ponies, cobs, mules, and donkeys of diverse body weights.

Anti-inflammatory Dosage. The preferred daily anti-inflammatory dose for humans is three or four capsules (based on capsule size) capsule, once or twice daily, possibly with dose splitting, every day for long-term use with chronic conditions; up to doubling the number of capsules, twice daily, are recommended for short-term administration (e.g. 30-90 days) for acute infections, injury, acute pain, or during recovery from chemotherapy. This recommendation is based on a reference body weight of 150 pounds (˜70 kg) and may be adjusted accordingly. The preferred daily anti-inflammatory dose for dogs is one or two capsules per 25 pounds (˜11 kg) of body weight, administered as often as three times daily, dependent on breed size; lower doses are recommended for long-term use whereas short-term administration (e.g. 30-90 days) is recommended for acute infections, injury, pain, or during recovery from chemotherapy. The preferred daily anti-inflammatory dose for horses is one scoop per 1000-1200 pounds of body weight (˜500-550 kg), once daily, every day for long-term use; one scoop, twice daily, is recommended for acute infections, injury, pain, or during recovery from chemotherapy. The dose may be adjusted for draft or miniature horses, ponies, cobs, mules, and donkeys of diverse body weights.

Wound Healing Dosage. The wound healing formulation is administered similarly to the anti-inflammatory formulation, at higher doses adjusted for more severe or acute conditions, preferably under observation by a healthcare professional or veterinarian.

### Antioxidant Functionality

Reactive oxygen species (ROS) and related reactive nitrogen species (RNS) are a constant threat generated from environmental exposures and as byproducts of the normal metabolism of oxygen. ROS/RNS play central roles in the control and regulation of biological processes such as growth, the cell cycle, programmed cell death, hormone signaling, biotic and abiotic stress reactions and development. Cells produce ROS/RNS under physiological control but increasing ROS becomes pathological and leads to oxidative stress, biological macromolecular damage, and disease. Since the 1990s, when scientists began to understand that free radical damage was involved in loss of general wellness by inducing fatigue, vulnerability to infections, headaches, worsened eyesight, joint pain, wrinkles, and the early stages of inflammation, cancer, cardiovascular diseases, diabetes, liver diseases, nephrotoxicity, vision loss, neurodegenerative processes, memory loss, and a host of other conditions that worsen with aging, the public has become increasingly aware of their existence and health effects.

Antioxidants are molecules, present at very low concentrations in normal cells, that delay, control, or prevent, damaging oxidative processes of ROS/RNS. Under normal circumstances, the rate and amplitude of oxidant formation is balanced by the rate of their removal; however, loss of balance between pro-oxidation and antioxidation results in oxidative stress. A fundamental observation, at least in vitro, is that most antioxidants at high doses may convert to pro-oxidants. Antioxidants donate an electron to stabilize and neutralize them. Like a domino that refuses to fall, an antioxidant can stop the free radical chain reaction in its tracks. In donating an electron, the antioxidant itself becomes a free radical but, antioxidants are special in that they are not very reactive themselves and are quickly able to stabilize.

The market for antioxidants (not only natural dietary supplements) is quite large—US$ 1.4 Billion in 2022 and is expected to reach US$ 2.0 Billion by 2028, exhibiting a CAGR of 5.6% of during 2023-2028 (Natural Antioxidants Market Share, Size and Forecast 2023-2028 imarcgroup.com). Natural dietary antioxidant supplements represent a very competitive space where unique ‘superfoods’ and megadoses of vitamins (e.g. A, C, and E), minerals (e.g. Cu, Mn, Se, and Zn), and a plethora of phytochemical extracts and isolates have been in demand.

Paradoxically, even at high concentrations, most extracted and isolated antioxidant compounds are unabsorbed by the body and result in inadequate uptakes and utilization of antioxidant compounds before being excreted out of the body. The antioxidant compounds need to be solubilized within the gastrointestinal tract in order to effectively increase their bioaccessibility and bioavailability. While antioxidant extracts and isolates may be made bioavailable, their bioavailability is ultimately dependent on several factors that include: molecular structure that influences stability and solubility; matrix of a food, supplement, or therapeutic formulation that contains other substances to either enhance or inhibit absorption or that dictates physical and chemical relationships between those different components to influence the bioavailability of the antioxidants, and; digestion pathways and first-pass metabolism that also affect the bioavailability of compounds. Therefore, while these compounds have the potential to be bioavailable, their actual bioavailability may be widely variable under physiological conditions.

Eventually, a number of peer-reviewed studies revealed the untoward effects or lack of effects of these antioxidant supplements at high doses. For example, supplementing with high doses of beta-carotene may increase the risk of lung cancer in smokers, and supplementing with high doses of vitamin E may increase risks of prostate cancer and one type of stroke; very high doses of Vitamin C may lead to a variety of gastrointestinal issues. Some studies have found that Vitamin E, β-carotene, and possibly high doses of Vitamin A supplements may be associated with higher all-cause mortality. Megadoses of selenium and zinc may lead to various toxicity issues, but many are related to creating copper and iron deficiencies.

As a result of these findings, healthcare professionals now recommend consumption of whole fruits and vegetables for optimal natural antioxidant intake. On the surface, adequate consumption of fruits and vegetables appears to be an easy solution. This recommendation requires a minimum of five servings per day with seven to ten servings more likely necessary for health benefits. Most consumers are unable to meet the requirement due to palate, lack of convenience, and economic reasons. Even meeting the requirement in number may not be adequate to provide adequate levels of antioxidants, as modern plant breeding and agricultural practices have led to bigger, faster-growing, disease-resistant, sweeter, or milder-tasting fruits and vegetables no longer dependent on phytochemicals for proliferation and survival.

Recent reports on mechanisms of action of natural antioxidants highlight the fact that natural antioxidants are heavily metabolized in vivo, with significant reduction of their redox potential at the physiological level. Those authors observed a growing interest in the interactions of natural antioxidants and their metabolites with proteins in intracellular signaling cascades and modulation of the gut microflora. Currently, efforts with natural antioxidants are trending toward pursuit of intrinsic antioxidant enzyme studies, genetic modifications such as epigenetics and chromatin structural changes, effects of antioxidants on hormonal activity, effects of antioxidants on the intestinal microflora, anti-aging effects of fermented preparations, and combination therapies using the synergistic effect of natural antioxidants. The last area of emerging interest in antioxidants pertains to this application.

Formulations in accordance with the present invention are potently antioxidant through VC, RC, TC, and AC that together provide both shared and distinct phytochemical components to extend its range of effective oxygen radicals scavenging. VC was studied further for antioxidant activity based on chemical analyses, with phenolics consistently averaging 85 to 95 mg/g dry wt. after processing and, of this total, 40-50 mg being comprised of an unusual anthocyanin composition. Phenolics, with anthocyanins contributed to the present inventive formulations exclusively by VC, exert their antioxidant activity through two main mechanisms: by acting as hydrogen atom donors or as metal ion chelators. The antioxidant capacities of phenolics depends on the number and arrangement of hydroxyl groups, the nature of the substituents in the ring structures, ionization state, steric hindrance, and the stability of the resulting phenoxy radicals. Under certain experimental conditions, the anthocyanin, delphinidin-3-O-glucoside, had the highest activity at two pH values, followed by cyanidin-3-O-glucoside and pelargonidin-3-O-glucoside. They effectively scavenge various biologically relevant reactive oxygen species (ROS) and reactive nitrogen species (RNS), including peroxyl (ROO·), hydroxyl (HO·), and superoxide (O₂^{·-}) radicals, singlet oxygen (1O₂), and peroxynitrite (ONOO—).

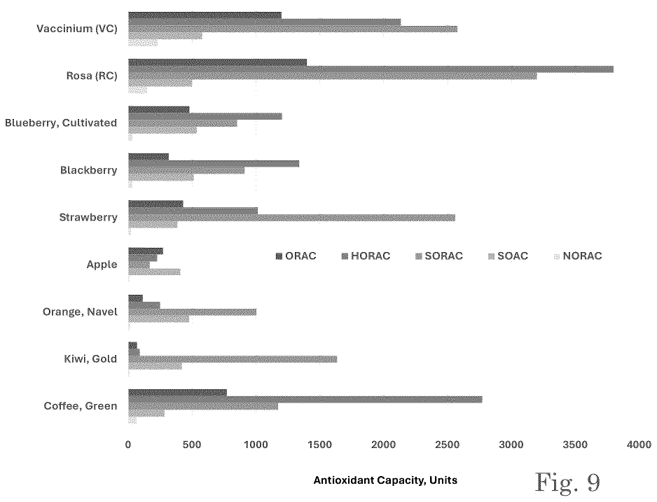

Initially, as reported in U.S. Pat. No. 8,591,964 B2, the oxygen-radical-absorbing capacity (ORAC) of VC was 1100-1200 Trolox equivalents per gram (TE/g) for its hydrophilic fraction and 45 TE/g for its hydrophobic fraction. This value was noted as very high for a whole fruit concentrate with only water removed, not an extract. Subsequently, ORAC 5.0 assays were used to measure a range of free radical species such as peroxyl, hydroxyl, superoxide anion, singlet oxygen, peroxynitrite, viz., ORAC, HORAC, SORAC, SOAC, and NORAC. The assays were conducted with a standardized protocol to provide comprehensive antioxidant activities using antioxidants covering both the hydrophilic and hydrophobic antioxidants present (Huang, D. J. Agric. Food Chem. 2005, 53,1841. doi:10.1021/jf030723c. Yang, J et al. Food Chemistry. 2014, 160, 338. doi:10.1016/j.foodchem.2014.03.059). VC successfully scavenges each type of radical consistently better than other samples, except for RC and green coffee for HORAC (FIG. 9). The presence of the top three out of six main anthocyanins for antioxidant activity is rare, and the ability to scavenge multiple radicals effectively is a highly desirable feature of a supplement ingredient. This result with ORAC 5.0 was unanticipated and supports the use of VC as the principal antioxidant in supplement formulation in accordance with the present invention.

RC, TC, and AC provide to the inventive formulations fortification of the phytochemical subclasses identified in VC or expand those known to have potent antioxidant activity that do not occur in VC. The ORAC of RC was found to be 1400-1500, compared to 1100-1200 in VC, and ORAC 5.0 of notable antioxidant capacity (FIG. 9). While both VC and RC contain ellagic acid, it is exceptionally abundant in RC; ellagic acid and ellagitannins are precursors of urolithin A, also important in the anti-aging effects linked to antioxidant activity. Catechin, present in VC, is very prominent in the composition of RC, along with important catechin subtypes such as epigallocatechin gallate (EGCG).

Gallic acid, copious in RC, is critical to the formation of catechin gallates, and is yet another intrinsically potent antioxidant demonstrated to effectively scavenge superoxide and hydroxyl radicals, and other ROS. RC contains lycopene, a noncyclic carotenoid, the most effective free radical scavenger in vitro of all non-xanthophyllic carotenoids. It increases levels of the intracellular antioxidant, glutathione, and stimulates the activities of antioxidant enzymes such as glutathione peroxidase (GSH), catalase (CAT), and superoxide dismutase (SOD). Lutein, another carotenoid found principally in TC and, to a lesser extent, in AC, is an effective suppressor of H₂O₂-induced ROS. TC and AC contain the powerful flavone antioxidant, luteolin and derivatives, and related apigenin, schaftoside, chrysoeriol, and their derivatives. Chrysoeriol is of interest because it is shown to markedly enhance the key transcription factor, nuclear factor erythroid 2-related factor 2(Nrf2 ) and antioxidant-associated genes (heme oxygenase-1 (HO-1) and NAD(P)H dehydrogenase [quinone] 1 (NQO-1), in particular); its protective effect against H₂O₂-induced oxidative stress presumably prevents mitochondrial dysfunction by upregulating antioxidant-related molecules. Other phenolics, including chlorogenic acid (in VC) and cis-caftaric acid (found in the flower element of TC and AC), the latter notable for significantly increasing SOD, CAT, and GSH while decreasing inducible nitric oxide synthase (iNOS).

In an unexpected move, results from strictly in vitro ORAC assays (i.e. those performed in chemical assays on extracts of foods) were withdrawn from a USDA database in 2012 due to a lack of physiological proof in vivo (humans) supporting the free-radical theory or that it provided information relevant to biological antioxidant potential (https://www.naturalproductsinsider.com/claims/usda-says-orac-tests-useless-removes-database-for-selected-foods). Dietary polyphenols were found to have little or no direct antioxidant food value following digestion, as the increase in antioxidant capacity of blood seen after the consumption of polyphenol-rich (ORAC-rich) foods is not caused directly by the polyphenols, but most likely results from increased uric acid levels derived from metabolism of flavonoids. Accordingly, regulatory agencies such as the Food and Drug Administration of the United States (FDA) and the European Food Safety Authority (EFSA) have published guidance forbidding food product labels to claim or imply an antioxidant benefit when no such physiological evidence exists. This guidance for the United States and European Union establish it is illegal to imply potential health benefits on package labels of products with high ORAC (“Guidance for Industry, Food Labeling; Nutrient Content Claims; Definition for “High Potency” and Definition for “Antioxidant” for Use in Nutrient Content Claims for Dietary Supplements and Conventional Foods”, U.S. Department of Health and Human Services, Food and Drug Administration, Center for Food Safety and Applied Nutrition, June 2008. “Scientific Opinion on the substantiation of health claims related to various food(s)/food constituent(s) and protection of cells from premature aging, antioxidant activity, antioxidant content and antioxidant properties, and protection of DNA, proteins and lipids from oxidative damage pursuant to Article 13(1) of Regulation (EC) No 1924/20061”. EFSA Panel on Dietetic Products, Nutrition and Allergies (2010). EFSA Journal. 8, 1489. doi:10.2903/j.efsa.2010.1489).

Nonetheless, these assays remain informative for comparison of different sources of peroxyl radical scavengers in vitro that correlate with more recently recognized bioactivities in vivo. Exogenous and endogenous antioxidants neutralize and/or prevent the damage caused to living cells by ROS/RNS. Because the Cellular Antioxidant Activity (CAA) assay accounts for some claims of uptake, metabolism, and location of antioxidant compounds within cells, it is a more biologically relevant method than the popular chemistry antioxidant activity assays. A number of alternative antioxidant assay kits now are commercially available (https://www.oxfordbiomed.com/tech-resources/oxidative-stress-best-practices/ antioxidants-and-their-measurement).

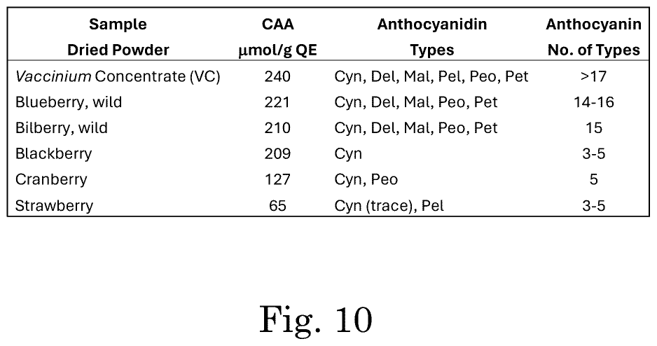

The CAA assay used here, essentially as described, quantifies the degree of intracellular protection provided by VC in comparison to other fruit powders and isolated phytochemical antioxidants against oxidative degeneration (Wolfe, K L and Liu, R H. J. Agric. Food Chem. 2007, 55, 8896. doi:10.1021/jf0715166; DCFDA/H2DCFDA—Cellular ROS Assay Kit (ab113851)|Abcam). More specifically, it uses dichlorofluorescein (DCF), easily oxidized to fluorescent dichlorofluorescein, as a probe to measure the ability of compounds to prevent the formation of DCF by 2,2′-azobis(2-amidinopropane) dihydrochloride (ABAP)-generated peroxyl radicals trapped within human hepatocarcinoma HepG2 cells. The decrease in cellular fluorescence when compared to the control cells indicates the antioxidant capacity of the compounds. The results are expressed in micromoles of quercetin equivalents per 100 micromoles of isolated phytochemical, micromoles of quercetin equivalents per 100 g of fresh fruit, or micromoles of quercetin equivalents per g of fruit extract. Among the pure compounds tested, quercetin (abundant in VC) has the highest CAA value, followed by kaempferol, epigallocatechin gallate (EGCG), myricetin, and luteolin; therefore, quercetin was used as the reference here. Historically, among the selected fruit powders tested, blueberry had the highest CAA value, followed by cranberry>apple=red grape>green grape. In this study by the present inventor, VC was found to be more effective as an intracellular antioxidant than wild blueberry, bilberry, blackberry, cranberry, and strawberry powders with values of 240, 221, 210, 209, 127, and 65 μmole quercetin equivalents per gram (QE/g), respectively (FIG. 10). Interestingly, VC, wild blueberry and bilberry contain delphinidins, whereas blackberry and cranberry contain only cyanidins, and strawberry only pelargonidins and a trace of cyanidin; this observation is consistent with in vitro results for delphinidin-3-O-glucoside, having the highest antioxidant activity, followed by cyanidin-3-O-glucoside and pelargonidin-3-O-glucoside.

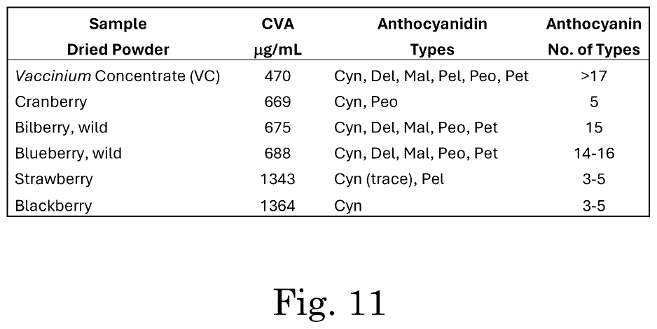

As an extension of cellular antioxidant activity, the protective effects of VC on cell viability and growth were evaluated in assays based on the bioluminescence of adenosine triphosphate, the primary energy unit for cells (Ahmann, F R et al. In Vitro Cell Dev. Biol. 1987, 23, 474. http://www.jstor.org/stable/4296094; Crouch, S P et al. J. Immunol. Methods. 1993. 15, 160, 81. doi:10.1016/0022-1759(93)90011-u). Used successfully for many years, a now commercially available assay, modified here, allows for the rapid, sensitive, and reproducible measurement of effects of purified phytochemicals or fruit powders on ATP levels in cells (https://www.abcam.com/products/assay-kits/luminescent-atp-detection-assay-kit-ab113849.html). HepG2 cells were seeded into each well, allowed to adhere, and treated for 4 hours with rotenone (25 μM), a naturally occurring isoflavone pesticide, rotenone plus test substance, and dimethyl sulfoxide (DMSO) solvent in glucose based complete media (https://faculty.ksu.edu.sa/sites/default/files/rotenone-induced_oxidative_stress_and_apoptosis_in_human_liver.pdf). After treatment, cells were lysed, exposed to the ATP substrate solution and signal was measured on a luminescent counter. Test compounds, either purified phytochemicals or fruit powders were added at doses between 100 to 1500 mg/mL, along with the positive control, rotenone, that induces cytotoxicity in HepG2 cells. A positive control determines the maximal expected signal in the absence of a test compound and DMSO, the negative control, is used to establish the background signal level. The values reported for test samples are the optimal concentration for overcoming the cytotoxicity of rotenone relative to DMSO control. VC was the most effective at 470 μg/mL, followed by cranberry, bilberry, wild blueberry, strawberry, and blackberry, at 669, 675, 688, 1343, and 1364 μg/mL, respectively (FIG. 11).

A large body of evidence suggests that ROS and RNS are important instigators of chronic diseases, many of which are related to DNA damage, inflammation, and the overall pathophysiology of aging. Previous studies have shown that when mammals age, the expression of Sirtuin1 (SIRT1) protein, a NAD-dependent deacetylase, decreases; induction and activation of SIRT1 has been associated with reduction of age-related diseases and extended lifespan. These studies have triggered the search for SIRT1 activators that may be used as dietary supplements to promote health and longevity. For evaluating VC influence on SIRT1 expression, HepG2 cells were incubated with and without VC, fruit powders, and isolated phytochemicals with documented antioxidant activity. After 18 hours of treatment, a fluorometric assay, designed for the rapid and sensitive evaluation of SIRT1 inhibitors or activators using crude SIRT1 fraction or purified SIRT1. The method was a minor adaptation of the SIRT1 Activity Assay Kit (Fluorometric) from Abcam to accommodate measurement of activity in lysates of treated cells (www.abcam.com/products/chip-kits/sirt1-activity-assay-kit-fluorometric-ab156065.html). SIRT1 expression and maximum percentage of the SIRT1 expression change after treatment are reported at the concentration that induced the maximum percentage of SIRT1 expression change. For VC, 44.4 μg/mL induced a maximum change in SIRT1 expression of 43.1%. This value appears low compared to those of pure phytochemicals, such as melatonin, carnosic acid, urolithin A, tetrahydrocurcumin, neochlorogenic acid, ferulic acid, quercetin, resveratrol, and astaxanthin, known to induce SIRT1 by one-to eight-fold. The increased expression of SIRT1 by these in various cell types occurs at concentrations in the 10⁻⁸ M-10⁻⁵ M range. In a whole fruit powder such as VC, the concentration of any SIRT1 activator is likely to be orders of magnitude lower. For example, the highest levels of resveratrol in highbush blueberries were found to be 140.0+/−29.9 picomole/g, comparable to the amount detected in VC, and that translates to approximately 6 femtomoles presented to this assay system (https://healthprofessionals.blueberry.org/research/resveratrol-in-raw-and-baked-blueberries-and-bilberries/). Notwithstanding this very low amount of resveratrol, VC and other Vaccinium species also contain quercetin, urolithin A, ferulic acid, and neochlorogenic acid, raising the possibility of additivity or synergy between these intrinsic phytochemicals for enhancing SIRT1 expression.

### Anti-Inflammatory Functionality

For use as an anti-inflammatory and immunomodulatory supplement (FIG. 6) the proposed antioxidant formulation with astaxanthin is further refined through addition of birch bark extract that typically contains 70-80 mg betulinic acid per g, added to achieve a final concentration of 1-2% by weight betulinic acid in the formulation. Quercetin, an anti-inflammatory, but multifunctional phytochemical, is added at a clinically relevant dose for humans. VC, RC, TC, and AC are incorporated at 70, 15, 5, and 10 percent each, respectively. The preferred amount per unit dose is 250-500 mg based on body weight using 70 kg human as a reference. The unit dose is administered with a meal one or two times daily, or split and administered with each meal and bedtime snack. For dogs, the reference unit dose is adjusted for breed body weight and administered according to feeding schedule. For horses, the reference unit dose is increased by a factor of eight and administered according to feeding schedule.

Antioxidant capacity is linked to SIRT1 and anti-inflammatory activity through many molecular entities and processes. The array of components that contribute to the remarkable antioxidant profile of VC are documented for anti-inflammatory effects, especially the phenolics, quercetin, resveratrol, and anthocyanins in a hydrophilic milieu. Astaxanthin powder, a potent lipophilic antioxidant and anti-inflammatory xanthophyllic carotenoid, reduces infiltration of inflammatory cells, inhibits the expression of proinflammatory cytokines, and increases the expression of anti-inflammatory cytokines. Astaxanthin and VC were found to have effects on the production of specific endothelium-derived immunoactive proteins in human umbilical vein endothelial cells (HUVEC). HUVECs are adaptable to many experimental objectives, but the conditions for their growth and treatment are situation-specific, as described in detail here. A wide variety of inflammatory factors may be measured in HUVECs; often focused on those associated with the development of cardiovascular diseases, these factors are involved in many other chronic conditions. Factors included monocyte chemotactic protein-1 (MCP-1 or CCL2), epithelial neutrophil-activating peptide-78 (ENA-78 or CXCL5), interleukin-6 (IL-6), interleukin-8 (IL-8 or CXCL8), interleukin-10 (IL-10), fibroblast growth factor (FGF), granulocyte colony stimulating factor (GCSF), and intercellular adhesion molecule (ICAM1). HUVEC is a well-regarded system to study inflammatory factors not only in humans but for extrapolation of these factors to other mammals, such as companion dogs and horses.

For this assay, VC and astaxanthin powder (2% by weight) either alone or in combination, were evaluated essentially as described (Zineh, I et al. Pharmacotherapy. 2006, 26, 333. doi:10.1592/phco.26.3.333). Human umbilical vein endothelial cells were obtained (Clonetics Cell Systems; Cambrex Bio Science Walkersville, Inc., Walkersville, MD). Approximately 5×10⁵ viable cells were seeded in endothelial cell growth-supplemented basal medium (EGM-2-endothelial cell medium-2; Cambrex Bio Science Walkersville, Inc.) and maintained at 37° C. under a 5% carbon dioxide atmosphere per manufacturer's protocol. The culture medium was changed 24 hours after seeding and every 48 hours thereafter until confluence. Cells were then treated with trypsin, seeded in a 48-well culture dish, and incubated under the conditions previously described. Cells used in these experiments were from the third passage. After cells reached 70-80% confluence in the culture dish, they were allowed to become quiescent in unsupplemented, serum-free medium for 24 hours before treatment.

When cells reached approximately 80% confluence, they were treated with VC (100 μg/mL) alone, astaxanthin powder (AX, 1 mg/mL of 2% by weight) alone, or VC+AX (100 μg/mL+1 mg/mL of 2% by weight) for 24 hours. VC, AX, and atorvastatin (LKT Laboratories Inc., St. Paul, MN), used as a reference compound (50 μM), were dissolved in dimethylsulfoxide (DMSO). DMSO treated cells served as negative controls. The final concentration of DMSO in conditioned media did not exceed 1% weight/volume. All experiments were performed in triplicate. After treatment, culture-conditioned media were collected, divided into aliquots, and stored at −80° C. for no longer than 7 days until multiplex measurement was performed for expressed protein content of chemokines, cytokines, and angiogenic factors.

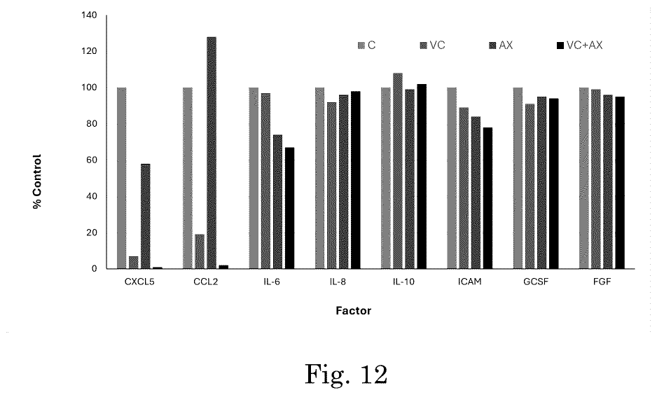

Flow-based immunofluorescence multiplex detection was employed to measure the culture-conditioned media protein content of CCL2, CXCL5, IL-6, IL-8, IL-10, FGF, GCSF, and ICAM. (Fluorokine MAP Human Cytokine Panel; R&D Systems Inc., Minneapolis, MN) with the Luminex 100 IS platform (Luminex Corp., Austin, TX). Concentrations for each sample were normalized against milligrams of total protein by using a standard bicinchoninic acid protein assay (Pierce Biotechnology, Inc., Rockford, IL). Standard curves of best fit for each analyte were generated by plotting the median fluorescence intensities of the analyte standards against their known concentrations by using Beadview multiplex data analysis software, version 1 (Upstate, Charlottesville, VA). Differences in biomolecular concentrations were compared by treatment group by applying one-way analysis of variance and a post-hoc Tukey's test as appropriate. Statistical analyses were performed with SPSS software, version 11.5 (SPSS Inc., Chicago, IL). Samples were tested in triplicate and experiments were conducted in duplicate. Standard deviations for each assay point were 1-3%+/−0.1-0.5% (p<0.0001 to p<0.05). Optimal results with VC were reported at a dose of 100 μg/mL (micrograms per milliliter), and 1 mg/mL for AX (whole algae powder standardized to 2% by weight or 20 μg astaxanthin per dose), determined in previous experiments. Compared to reference compound, atorvastatin, that significantly inhibits CXCL5, CCL2, IL-6, and IL-8, comparable reductions in expression of CXCL5, CCL2, and IL-6 were observed with VC, AX, and VC+AX (p<0.001 to p<0.05). Results are presented as a percent compared to the control (100%) in FIG. 12.

Anthocyanins, exceptionally abundant in VC, also are potent antioxidants and anti-inflammatory agents. VC inhibited CCL2 expression to 19% and VC+AX to 2%; conversely, AX increased MCP-1 expression to 28% higher than the control. This potent effect by VC is against CCL2, a pro-inflammatory cytokine released by various cell types in response to events such as oxidative stress, cytokine release, and growth factor release. CCL2 enhances the expression and production of other pro-inflammatory cytokines such as IL-1β, IL-4, IL-6, tumor necrosis factor-α (TNF-α), transforming growth factor-β (TGF-β), lipopolysaccharide (LPS), interferon gamma (IFN-γ), platelet-derived growth factor (PDGF), vascular endothelial growth factor (VEGF), macrophage colony-stimulating factor (M-CSF), and granulocyte-macrophage colony-stimulating factor (GM-CSF). CCL2 plays a significant role in the migration of monocytes and T cells to an inflammatory locus. New data suggest that the functions of CCL2 extend in scope beyond its original characterization as a chemoattractant and that it impacts leukocyte behavior, influencing adhesion, polarization, effector molecule secretion, autophagy, killing, and survival. The direction of these CCL2-induced responses is context dependent and, under certain conditions, synergistic with these other inflammatory stimuli. The involvement of CCL2 signaling in multiple conditions, such as autoimmune diseases including rheumatoid arthritis and multiple sclerosis, as well as infectious diseases, and inflammatory diseases including atherosclerosis, obesity, diabetes, and various types of cancer renders it an interesting therapeutic target.

Despite effort, current inflammatory factor targeting strategies have not met early expectations in the clinic. The data presented here with VC and VC+AX are corroborative with those obtained with a multi-species fruit mixture comprised of blueberry (Vaccinium angustifolium Ait. fruit), strawberry (Fragaria chiloensis (L.) Mill. fruit), cranberry (Vaccinium macrocarpon Ait. fruit), bilberry (Vaccinium myrtillus L. fruit), elderberry (Sambucus nigra L. fruit), and raspberry (Rubus idaeus L. seed) extracts and powders (Bagchi D et al. Biochemistry (Mosc). 2004, 69, 75 (1 p preceding 75). doi: 10.1023/b:biry.0000016355.19999.93). All fruit preparations together were necessary to capture four of the six major anthocyanidins, malvidin, cyanidin, delphinidin, and petunidin, and respective anthocyanin derivatives; notably, peonidin and pelargonidin are unreported in the commercialized formula composition (https://www.pureencapsulationspro.com/media/Optiberry.pdf). More importantly, all fruit preparations together were required to achieve a comparable inhibitory effect of VC or VC+AX on CCL2. The results with VC or VC+AX suggest promise to address therapeutically CCL2.

The most dramatic inhibition by VC was of CXCL 5 expression to 7%, AX to 58%, and VC+AX to 1%. CXCL 5 is produced by cells in response to IL-1β and TNF-α that are produced previously by cells in response to CCL2. While CXCL5 is not a pro-inflammatory cytokine per se, it plays a role in inflammation by promoting the release of other pro-inflammatory cytokines, such as IL-8, that is produced by macrophages and other cell types. The potent inhibitory effect of VC on CXCL5 was unanticipated, but astaxanthin has been shown to inhibit CXCL5 gene expression in a microarray assay but not expression in a living system such as HUVECs (Mao, K et al. Aging (Albany NY) 2020,12, 18716. doi:10.18632/aging.104042).

An inhibitory effect on CXCL5 expression also is observed with an extract of Skeletonema marinoi (20 μg/mL) that contained fucoxanthin, a xanthophyll structurally related to astaxanthin (Calabrone, L et al. Cells. 2023, 12, 1053. doi:10.3390/cells12071053). Although elevated levels of CXCL5 are linked to myriad inflammatory conditions and to stimulation of angiogenesis, of interest here is its early role in inflammatory responses to sunburn from UV exposure (https://www.sciencedaily.com/releases/2011/07/110706144612.htm). Dysregulated CXCL5 participates in tumor metastasis and angiogenesis in human malignant tumors, promotes tumor formation by triggering the migration of immune cells to tumors, and promotes immunosuppressive characteristics of the tumor microenvironment. In addition, CXCL5 also promotes tumor cell metastasis and recruits vascular endothelial cells for angiogenesis. All these functions are integral to many tumors. Any potential therapies against the involvement of CXCL5 in the progression of any tumor is highly desirable.

VC inhibited IL-6 expression to 97%, AX to 74%, and VC+AX to 67% and ICAM expression to 89%, AX to 84%, and VC+AX to 78%. IL-6 acts as a pleiotropic cytokine that plays multiple roles in immune responses, inflammation, hematopoiesis, bone metabolism, embryonic development, and other fundamental processes. It has both pro-inflammatory and anti-inflammatory effects. In a pro-inflammatory context, IL-6 plays a role in the recruitment of neutrophils to sites of inflammation. Its anti-inflammatory regulatory effects include maintenance of body temperature, bone health, brain function, and homeostasis.

ICAM is a protein that is expressed on the surface of many cell types, including endothelial cells, leukocytes, and platelets, and plays a crucial role in inflammation by facilitating the adhesion and transmigration of leukocytes across the endothelium into inflamed tissues. ICAM ligation produces pro-inflammatory effects such as inflammatory leukocyte recruitment by signaling through cascades involving several kinases.

Fiinally, for IL-8, FGF, GCSF, and IL-10, every treatment insignificantly affected expression by </=10% of control (FIG. 9). Specifically, VC inhibited expression to 92%, AX to 96%, and VC+AX to 98% of IL-8, a pro-inflammatory cytokine. The potent growth factors, FGF and GCSF, were insignificantly decreased by all treatments. FGF expression was inhibited by VC to 99%, AX to 96%, and VC+AX to 95%. VC inhibited GCSF expression to 91%, AX to 95% and VC+AX to 94%.

Of all the factors evaluated, expression of anti-inflammatory cytokine IL-10 was modestly enhanced by VC, but unaffected by AX and VC+AX. IL-10 expression was increased by VC and VC+AX to 108% and 102% of the control, respectively, whereas AX inhibited expression to 99%. IL-10, a cytokine with multiple, pleiotropic effects in immunoregulation and inflammation, suppresses the production of pro-inflammatory cytokines such as IL-1, IL-6, IL-8, GCSF, GMCSF, and TNF-α to help control inflammation, autoimmune responses, and angiogenesis. It downregulates the expression of Th1 cytokines, MHC class II antigens, and co-stimulatory molecules on macrophages. Moreover, it suppresses macrophage synthesis of reactive oxygen intermediates and nitric oxide and blocks certain cyclooxygenase-2-dependent biosynthetic targets. IL10 enhances B cell survival, proliferation, antibody production, and supports wound healing.

Taken together, results of these experiments in FIG. 12 support the inclusion of VC and astaxanthin into the inventive formulation for anti-inflammatory activity. A few examples of components of VC that likely have contributed to these results and would be complemented by other concentrates in the inventive formulation are: stilbenoids, resveratrol and pterostilbene (VC), flavonols, quercetin (mostly in VC and RC), rutin (VC), and kaempferol (RC); flavanols, like EGCG (RC), flavones, apigenin and luteolin (TC and AC). Those that inhibit the expression and activation of IL-6 are quercetin, kaempferol, EGCG, and apigenin. IL-8 expression and activation is modulated also by kaempferol, quercetin, and related flavonols, fisetin (found in apples, another Rosa species) and chrysin, and by exerting a strong inhibitory effect on TNF-α-induced IL-8 promoter activation. EGCG increases the amount of IL-10 in the hippocampus to reduce neuroinflammation and luteolin down-regulates the secretion of IL-6, IL-1β, and TNF-α. Kaempferol, chrysin, apigenin, and luteolin are active inhibitors of ICAM protein expression and regulate ICAM gene expression. Little has been reported in the literature about direct effects of flavonoids on FGF or GCSF, consistent with lack of observed effects in HUVECs. On the other hand, betulinic acid is shown to have complementary effects on those observed here with VC and astaxanthin, supporting its inclusion with core matrix for anti-inflammatory effects (https://www.frontiersin.org/journals/pharmacology/articles/10.3389/fphar.2022.883857/full).

### Wound Healing Functionality

Any physical, chemical, thermal, microbial, or immunological action on living tissue may result in disruption of a cellular, anatomical, and functional continuity of the living tissue, thereby creating a wound. This may range from a simple beak in the epithelial integrity of the skin or it may be deeper, extending into the subcutaneous tissue with damage to other structures such as tendons, muscles, vessels, nerves, parenchymal organs, and bones, as elaborated below.

Based on the underlying cause of wound creation, wounds may be classified into two main groups: open and closed. Open wounds bleed and are susceptible to invasion by microorganisms, whereas closed wounds (e.g. contusions, hematomas, and crush injuries) are not. These categories also are defined as penetrating or non-penetrating, external or internal, based on the wound origin. Internal wounds result from impaired immune and nervous system functions and/or decreased supply of blood, oxygen, or nutrients to that area, such as in cases of chronic medical illness (e.g. diabetes, atherosclerosis, and deep vein thrombosis). A particular case of an internal wound that bleeds and is associated with infection is a gastric ulcer. Depending on the healing time, wounds are further classified as acute or chronic. Acute wounds that heal uneventfully with no complications in the predicted amount of time are considered clean, while chronic wounds take a longer time to heal and might have some complications. Wound healing of all types is a complex process that involves four sequential but partially overlapping phases. This sequence is observed typically with an external, open wound, but occurs to different extents in others without visible evidence.

The initial stage is hemostasis or wound closing through blood clotting. Blood vessels constrict to limit blood flow, platelets (also called thrombocytes) adhere to seal the vessel rupture, and coagulation reinforces the platelet plug with fibrin threads. Hemostasis occurs rapidly, preventing excessive bleeding, but also begins to prevent infection. Platelets, despite being fragments of cells lacking a nucleus, can recognize pathogenic microorganisms and secrete various immunoregulatory cytokines and chemokines, thus facilitating a variety of critical immune effects and regulatory functions. The second stage is inflammatory during which injured blood vessels leak transudate (a mixture of water, salt, and cellular proteins), to effect localized swelling to clean and disinfect. After initiation of inflammation by platelets, the most prevalent and active cells in this stage are macrophages, as they are in all stages, sometimes being referred to as the orchestrator of the healing process, along with fibroblasts and endothelial cells. Macrophages, accompanied by neutrophils and leukocytes, clean the wound by engulfing debris and destroying it through a process called phagocytosis that also occurs during this stage. The third stage of proliferation involves the growth of new tissue to replace the damaged tissue. Hemoglobin and oxygen-rich red blood cells arrive at the site to ensure the granulation tissue is healthy and receives sufficient oxygen and nutrients. During the third stage of rebuilding, fibroblasts lay the foundation for proliferation of new tissue composed of collagen and extracellular matrix (ECM) proteins. The wound contracts as new tissues form, and a network of blood vessels develops (angiogenesis) to nourish the granulation tissue with a pink or red color and uneven texture if healthy. In the final maturation stage, tissue strength and function are restored; collagen fibers reorganize and, as the wound matures, scar tissue forms but becomes gradually less noticeable over time.

On the molecular level, chemokines are expressed by platelets, as well as those secreted by macrophages (and endothelium) are key regulators of the wound healing process from the beginning and instruct cells around the wound to conclude by making the structural protein, collagen, for tissue rebuilding and remodeling. Stage-related, pro-inflammatory cytokines secreted are CCL2, CCL5, CXCL1, CXCL2, CXCL8 during inflammation, and CCL2 and CCL3 during proliferation; angiostatic chemokines, CXCL10 and CXCL11, are secreted during maturation/remodeling. CXCL5 has several interactive roles with other signaling molecules throughout the wound healing process. CXCL5 signals through the chemokine C-X-C motif receptor 2 (CXCR2 ) in a signaling axis that activates the epithelial-mesenchymal transition (EMT). EMT is important in wound healing because it facilitates the transformation and mobility of cells, aids in the re-epithelialization of tissue, mediates the inflammatory response, and helps restore tissue integrity. CXCL5 is speculated to interact with other factors released by endothelial cells such as vascular endothelial growth factor (VEGF) and chemokines CCL2 and CCL5 and CXCL1, CXCL8, CXCL9, CXCL10 and CXCL11 to promote angiogenesis and leukocyte migration, working together to coordinate the wound healing process. CXCL5 directly up-regulates IL-1β/IL-6/tumor necrosis factor-α and down-regulates claims of VEGF signaling through CXCR2. Data regarding a given phytochemical effect on specific cytokines/chemokines or molecular processes is relatively sparse.

The normal physiology of wound healing at the molecular level depends on low levels of ROS/RNS and oxidative stress but depending on the type and severity of the wound, ROS/RNS levels may become abnormally high. Polyphenolics, such as diverse flavonoids exhibit antioxidant and anti-inflammatory effects and help to stabilize blood vessels during the initial clotting process. Of these, quercetin, is a potent antioxidant and anti-inflammatory effects; it supports platelet function to help prevent excessive bleeding. Quercetin and epigallocatechin gallate, genistein, and resveratrol impact other signaling pathways, including that mediated by transforming growth factor-beta (TGF-β) and the mitogen-activated protein kinase pathway.

Non-flavonoid polyphenols also play a role in wound healing both as antioxidants and anti-inflammatories, but also through different mechanisms. Anthraquinones (e.g. aloin and emodin found in aloe vera) are well-known, as are astringent tannins that aid in vasoconstriction, reducing blood flow and promoting clot formation. Triterpenes possess antimicrobial and antioxidant effects and contribute to the reparative process during wound healing whereas carotenoids restore the skin epithelial layer and tissues. Phytosterols exhibit antimicrobial and anti-inflammatory effects. Polysaccharides, such as glucomannan, acetylated polymannan, acemannan, mannose-6-phosphate, and inulin help to create a favorable environment for tissue regeneration. Vitamins E and C play crucial roles in wound healing with Vitamin E being essential for tissue repair, while vitamin C supports collagen synthesis and general immune function.

In many traditional medicine systems, plants that contain these bioactive phytochemicals have a long and rich history of use to promote clotting and arrest bleeding. The genera with cosmopolitan use are Solidago, Calendula, Plantago, Rumex, and Symphytum. Species from warmer climates, such as Curcuma, Terminalia, Centella, Bidens, Aloe, and Rauwolfia have confirmed wound healing activity. In Alaska, the most popular, even to this day, include Capsella bursa-pastoris (L.) Medik. (shepherd's purse), Potentilla L. species (such as marsh fivefinger, cinquefoil, and silverweed), Pyrola L. species (wintergreen), Urtica L. species (nettle), Sanguisorba canadensis L., syn. Sanguisorba stipulata Raf. (burnet), Solidago L. species (goldenrod), Geranium erianthum DC. (woolly geranium) and Geranium Bicknelli Britton (Bicknell's cranesbill), Heuchera glabra Willd. ex Roem. & Schult. (alpine heuchera), Polygonum bistorta L. (pink plumes), and Rumex crispus L. (yellow dock); however, the most widely recognized in Alaska and worldwide is the genus Achillea L. (https://alaskaherbalsolutions.com/11-plants-that-will-stop-bleeding/).

Thus, Achillea, with its valuable phytochemical profile and a long history of use for wound healing, is desirable for inclusion in core matrix for a wound healing formulation. Achillea and Rosa are used frequently in topical preparations for skin irritation and to prevent scarring. Taraxacum, owing to its rich content of vitamins (especially vitamin C, like Rosa), minerals, and other bioactive compounds supports overall health, which indirectly contributes to wound healing. Addition of Betula to the wound healing formulation is supported by a notably extensive literature (Ishfaq, B et al. Front. Bioeng. Biotechnol. 2023, 11. https://doi.org/10.3389/fbioe.2023.1042077).

Vaccinium species, despite their copious polyphenolics and well-documented antioxidant properties, are rarely cited for value in any of the wound healing phases. Insights into the molecular basis of the wound healing process argue against inclusion of VC and astaxanthin in the core matrix formulation for this purpose, especially in Phase 2 (acute inflammation), due to their inhibitory activity against CCL2 and CXCL5 demonstrated here in HUVECs, despite many benefits suggested in related V. myrtillus (Sharma, A and Lee, H-J. Curr. Issues Mol. Biol. 2022, 44, 4570. doi:10.3390/cimb44100313). As is typical with antioxidants and anti-inflammatories, opposite effects from those predicted may occur in practice due to context. VC appears to stimulate platelet recovery, as far as numbers, but it does not appear to affect their ability to produce chemokines and cytokines either (such as CCL2 and CXCL5). Astaxanthin also is a good example, demonstrated here and by many others, as it helps to reduce inflammatory factor production in HUVECs; simultaneously, however, it activates T-cells and natural killer (NK) cells to boost immune function in vivo.

The effect of plants on platelet numbers, instead of their recognized influence on clotting, has received considerable recent attention. Having less than the normal number of platelets is known as thrombocytopenia; having more than the upper normal limits of platelets is a condition called thrombocytosis. The principal causes of thrombocytopenia are deficient production of platelets by megakaryocytes in the bone marrow, increased breakdown of platelets in the bloodstream, and increased breakdown of platelets in the spleen or liver. The above conditions may result from viral and bacterial infections, heavy alcohol consumption, genetic syndromes, autoimmune disease, swollen spleen, and chemotherapy drugs. When quantified in humans, a normal platelet count ranges from 150,000-450,000 platelets per microliter of blood. Normal platelet count in dogs is >175,000-500,000 platelets per microliter of blood, whereas normal platelet count in horses is >100,000-400,000 platelets per microliter of blood, among the lowest in mammalian species.

Various plants appear to promote platelet number recovery, but one that is cited as undesirable for this purpose is cranberry (known botanically as Vaccinium macrocarpon Aiton, a taxonomic cousin of Vaccinium in VC (https://www.verywellhealth.com/how-to-increase-your-platelet-count-5190448). Another article specifically cites blueberries, presumably Vaccinium corymbosum L., as a good source of quercetin, to be avoided for recovery of platelet counts (https://www.verywellhealth.com/foods-to-eat-and-avoid-with-itp-5206758).

### Example 4—Case Study of Platelet Recovery in Elderly, Human Cancer Patient

A case study was initiated at the request of a lay person to administer VC to his elderly mother-in-law suffering with chronic lymphocytic leukemia (CLL) as part of a post-cancer recovery regimen. The 83-year-old female presented to a transfusion clinic in British Columbia with chronic lymphocytic leukemia and post-chemotherapy fatigue and weakness. Treatment was with fludarabine, cyclophosphamide, and rituximab (FCR) therapy for six 28-day (4 weeks) cycles, the upper limit of tolerability for this patient. FCR resulted in refractory thrombocytopenia (grade 2) and neutropenia (grade 2). Prior to chemotherapy, the patient's baseline platelet count was approximately 300,000/μL. Due to a rapid decline from healthy levels of platelets in the patient and unresponsiveness of platelet recovery over the 6 weeks following FCR conclusion, despite initiation of transfusions, the patient's family sought supplement support for her thrombocytopenia.

The patient was required to undergo a total of six platelet transfusions. The nadir of her platelet count during the first month was ˜37,000/μL and counts did not move from ˜50,000/μL after administration of five transfusions. The decision was made to trial VC after four transfusions after that lowest count. At Week 6.5, a daily dose of 400 mg of VC, split to 100 mg TID with meals and 100 mg with a bedtime snack, was administered to encourage platelet count recovery. By Day 3 of VC use, the patient's platelets rose to ˜83,000/μL and continued to rise to achieve a count of ˜125,000/μL at Week 9. Thereafter, the counts fell again and fluctuated between 60,000/μL to 95,000/μL until she received the sixth transfusion at Week 14.5 and experienced a rapid rise in counts to ˜118,000/μL in the next two days. Thereafter, they began to fall again, and VC administration was revised at Week 15.5 to include AC, with 400 mg of VC:AC (3.5:0.5), split to 100 mg TID with meals and 100 mg with a bedtime snack. By Day 3 after revision, counts recovered from ˜100,000/μL to ˜118,000/μL again and continued to rise to ˜240,000/μL by Month 6.

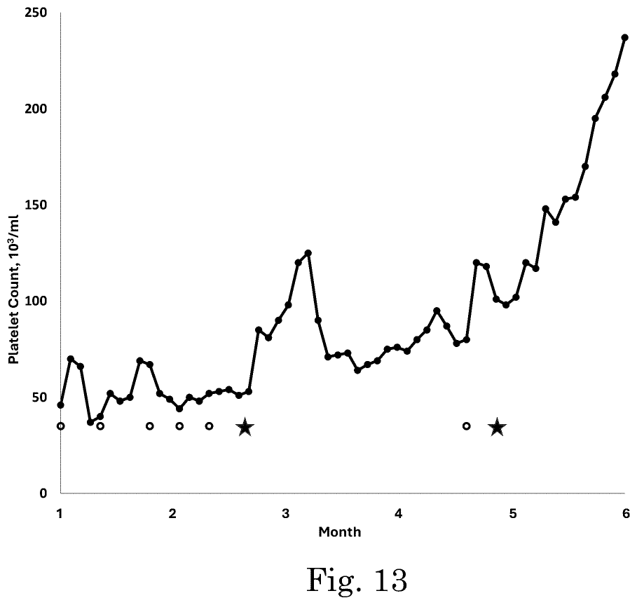

FIG. 13 depicts the trend in the patient's platelet count over six months after starting treatment, from December 2021 through June 2022. The patient demonstrated compliance for the duration of her recommended treatment and continued using it from June 2022 until follow-up with her son-in-law on March 2023. Additionally, she reported no side effects and did not display any adverse toxicities with VC use. No species of Vaccinium is known to directly or independently stimulate platelet recovery. In addition to the obvious benefit to cancer patients who need to clot more effectively to prevent excessive bleeding and initiate wound healing, less frequent transfusion administration is highly desirable.

This was an unanticipated observation with VC, an excellent source of quercetin and its derivatives, in direct contradiction to these recommendations according to bloodwork data on a post-chemotherapy cancer patient. The results here resemble more closely those reported for Carica papaya L. (papaya) leaf extracts (CPLE) on a gliobastoma multiforme (GBM) patient (Koehler, A et al. Integr. Cancer Ther. 2022, 21:15347354211068417. doi:\10.1177/15347354211068417). This extract of C. papaya is being considered as a far more inexpensive means of ameliorating thrombocytopenia compared to FDA approved recombinant interleukin-11 (rhIL-11) and a variety of treatment options like platelet transfusion, splenectomy, and platelet management with various types of corticosteroids (Mishra, K P et al. Indian J. Clin. Biochem. 2023, 38,297. doi:10.1007/s12291-022-01097-x).

Thrombocytosis may be caused by a variety of factors and is classified as essential or reactive. The cause of essential thrombocytosis appears to be connected to changes in certain genes. The bone marrow produces too many of the cells that form platelets, and these platelets often malfunction. This poses a much higher risk of clotting or bleeding complications than reactive thrombocytosis. Causes of reactive thrombocytosis are numerous and include viral infections, surgery and other types of traumas, large spleen/spleen removal, blood loss, folate and iron deficiencies, hemolytic anemia (often due to certain blood diseases and bone marrow diseases, such as aplastic anemia, disseminated intravascular coagulation, Vitamin B12 deficiency anemia, and leukemia), or autoimmune disorders (such as thyroid disease, lupus, sarcoidosis and scleroderma), other inflammatory disorders (such as inflammatory bowel disease, rheumatoid arthritis, and cancer), and kidney or liver disease. Platelets play an important role in cardiovascular disease both in the pathogenesis of atherosclerosis and in the development of acute (often fatal) thrombotic events. Modifications in the functioning of the hemostasis system result in the development of serious and even fatal complications including myocardial infarction, ischemic stroke, and deep vein thrombosis, which are associated with thromboembolism of affected vessels. The goal of antithrombotic therapy is to reduce the risk of thromboembolic events by lowering platelet activity, inhibiting the coagulation system, and improving endothelial function; however, this aim often is not achievable due to adverse drug reactions or drug resistance.

Therefore, a search for new substances of plant origin with beneficial effects in hemostasis is underway by researchers. A subclass of phenolics, tannins, has been used throughout history for their ethnopharmacological properties in thrombotic disorders. Among best-studied plants in traditional medicine with the potential to prevent thromboembolic events are tannin-rich species in the genera Acacia, Agrimonia, Camellia, Geranium, Rhus, Hamamelis, Krameria, Lythrum, Phyllanthus, Potentilla, Quercus, Rubus, Sanguisorba, and Terminalia. Tannins are considered by some to be the most relevant from a medicinal point of view for clotting; however, one review discusses the varying influences of tannins on particular components of hemostasis, namely platelets, coagulation system, fibrinolysis system, and endothelium, under several in vitro and in vivo conditions. Results observed in several studies with the evaluation of tannins in animal models of thrombosis open new perspectives on plants, not only for clotting, but as a source of thrombolytic agents (Marcińczyk, N et al. Front. Pharmacol. 2022, 12. https://doi.org/10.3389/fphar.2021.806891).

Notably, none of the species used to make core matrix concentrates is cited on the list of the most relevant for this purpose although all are known to contain tannins and, of those, Achillea is a rich source with ethnobotanical evidence (Smoilovska, H P et al. Curr. Issues Pharm. Med. 2023, 16, 130. https://doi.org/10.14739/2409-2932.2023.2.281344). While yarrow is famous for arresting bleeding in the early stages of wound healing, high doses of yarrow may slow down blood clotting; if taken with medications that thin the blood, such as aspirin, clopidogrel (Plavix), and warfarin (Coumadin), it may raise the risk of bleeding (https://www.rxlist.com/supplements/yarrow.htm). Another specific ellagitannin, rugosin, has been identified in in the leaves of R. acicularis and R. rugosa the principal and minor species in RC, but not the hip organ included in the concentrate (Olennikov, D N et al. Plants (Basel). 2021, 10, 2525. doi:10.3390/plants10112525). Among the nine known ellagitannins, rugosin E was the most potent platelet aggregating agent; aggregation lowers effective circulating platelets, slowing blood clotting.

### Example 5.—Use of RC and AC for Platelet Disorders

As a corroboration for use of core matrix for platelet disorders, an alternative and complementary medicine specialist requested separate components to test on his patients diagnosed with different types and severity of thrombocytopenia or thrombocytosis. He claims that 400-500 mg/day (based on body weight) with VC:RC:TC:AC of 3.0:0.5:0.25:0.25 or VC:RC:TC:AC up to 4:0:0:0 with the latter being preferred, especially for immediately post-chemotherapy, thrombocytopenia patients. This recommendation was consistent with observations on the 83-year-old female transfusion patient.

For thrombocytosis patients, he initiated therapy at 400-500 mg/day with ratios of VC:RC:TC:AC of 0:2.65:0:1.35. to VC:RC:TC:AC of 0.125:2.5:0.125:1.25. In both cases, unit doses of 100-150 mg may be split TID, with meals, and bedtime snacks. Eventually, a ratio of double RC: AC proved to provide the best results. For maintenance or to prevent recurrence, reduced doses and adjustments depending on follow-up results and individual parameters have been used safely and with success.

Although no species of Vaccinium is known to directly or independently stimulate platelet recovery, or adjust their counts in thrombocytosis, a recent report pertains to the overall wound healing capacity of Vaccinium angustifolium Aiton (Maine wild blueberry). The researchers showed that an extract enriched in phenolics improved vascularization and cell migration, a critical step in the healing process, in human umbilical cord cells (HUVEC).

Subsequently, they showed that comparing animals treated with a base gel that did not contain the phenolic extract to a control group that received no treatment, the group treated with the blueberry extract showed improved migration of endothelial cells to the wound site. They also experienced a 12 percent increase in wound closure. The conclusion was made that wild blueberries have the potential to enhance cell migration, new blood vessel formation (angiogenesis) and vascularization, and to speed up wound closure. This is especially important in conditions that require enhanced wound closure in patients with chronic wounds such as diabetic wounds, burns and pressure ulcers (https://studyfinds.org/blueberries-hard-to-heal-wounds/).

### Example 6—Thoroughbred Stallion With Internal and External Wounds

In a pilot study, a 14-year-old, 1100-pound, thoroughbred stallion participated in a study to evaluate safety and palatability of the formulation. At that time, medical concerns stemmed from a congenital gastric ulcer and history of gastrointestinal issues, principally due to stomach and foregut ulcers and possible hindgut imbalances. Anxiety and episodic aggression, typical behaviors of stallions were observed, but more likely were due to abdominal pain. The horse had been observed to nip routinely at his side, near the abdomen. At some time during February 2023, he nipped hard enough to cause a subcutaneous mass of approximately 4 inches by 2.5 inches, found to be detached with palpation by the veterinarian. The owner believes this behavior is a result of chronic abdominal pain from congenital ulcers.

Prior to commencement of formulation administration, bloodwork was reported on Jul. 15, 2023. All parameters were within normal range, except three. The first was hypocalcemia, with a value of 10.6 mg/dL (normal range 10.8-13.5 mg/dL). The second was a neutrophil count of 4868/mL (normal range 2700-7000/mL) and lymphocyte count of 1704/mL (normal range 1500-5500/mL) that yielded a neutrophil to lymphocyte ratio of 2.86:1, higher than the optimal ratio of approximately 2:1. The third was elevated lactate dehydrogenase (LDH), with a value of 409 IU/L (normal range 81-390 IU/L).

Beginning on Aug. 1, 2023, the horse was administered core matrix with added birch extract, astaxanthin, quercetin, piperine, and various functional fillers including apple peel powder, carrot, cranberry, and watermelon. As of Aug. 7, 2023, the owner reported that horse was eating the formulation, once per day, and showed no untoward effects. On, or about, Aug. 17, 2023, the horse presented with a contusion and large hematoma on the left side that was ascribed to a pasture accident. A few days later, the wound opened on its own causing a substantial area of fluid drainage prompting the owner to administer one course of oral sulfamethoxazole; the hematoma and wound resolved quickly by the time the formulation was complete on Aug. 28, 2023.

As of Sep. 4, 2023, the owner reported that overall abdominal nipping and discomfort appeared less while the horse consumed the formulation, but in the week since running out, nipping had increased again. Also, on Sep. 4, 2023, the veterinarian returned to draw blood as follow-up to the first round of formulation administration.

After the first month (reported Sep. 4, 2023), of formulation administration hypocalcemia had resolved at 11.8 mg/dL (normal range 10.8-13.5 mg/dL). Lactate dehydrogenase was now in normal range with a value of 220 IU/L (normal range 81-390 IU/L). The neutrophil count of 4160/mL (normal range 2700-7000/mL) and lymphocyte count of 1664/mL (normal range 1500-5500/mL) yielded an improved neutrophil to lymphocyte ratio of 2.5. The owner reported that the horse nipped at its side less frequently and that the open wound had healed without further medical intervention. In general, the horse appeared to experience less pain and anxiety and was less aggressive. At that time, the owner and veterinarian agreed to continue administration of formulation for unrelated melanoma therapy and general wellness.

### Anti-Tumor Functionality

Exceptional amounts of certain, highly regarded antitumor agents are present in VC, such as anthocyanins (i.e. delphinidins) and proanthocyanidins (i.e. Type A2). In TC, taraxerol has also shown significant anti-inflammatory activity and, with taraxasterol, are important anti-tumor agents (Jiao, F et al. Front. Pharmacol. 2022,13, 927365. doi:10.3389/fphar.2022.927365). TC and AC contain apigenin and luteolin, both potent anti-tumor flavones, among others.

For use against osteosarcoma and related malignancies, VC, RC, TC, and AC are used in equal parts with enough birch bark extract to deliver 10-30 mg/g betulinic acid per dose. To this, 50-100 mg of artemisinin is added, with the lower dose is preferred for small-to medium-sized dogs and the higher dose for large-to giant-sized dogs. Either dose is administered twice daily with food for at least one month, continued for up to 6 to 12 months at a time (New Hope for Treating Cancer-Whole Dog Journal https://whole-dog-journal.com). A more refined dose of 2.2-4.4 mg of artemisinin per pound of body weight. Alternatively, artemether may be substituted for artemisinin and given two times a day, 12 hours apart (https://www.dogcancer.net.au/dog-cancer-osteosarcoma-bone-cancer.php) or a dose of 1 milligram of artemether per kg body weight per day, preferably given with along with butyrate and vitamin D-3, according to the same feeding schedule (Could This Plant Be A Canine Cancer Killer? (dogsnaturallymagazine.com) https://www.dogsnaturallymagazine.com/artemisinin-and-canine-cancer/). The recommendation to separate artemisinin from a meal of iron-rich food is recommended due to its reactivity with the mineral (Natural Cancer Treatment For Dogs-Dogs Naturally (dogsnaturallymagazine.com). However, adequate dietary iron is recommended not only to maintain general health, but because tumor cells that typically have elevated intracellular iron levels are more sensitive to artemisinin than normal cells. Any dog treated with radiation therapy or for seizures should not receive this therapy. Although the combinations are not contraindicated with conventional therapies, a veterinarian should be consulted prior to administration.

Preparations of outer birch bark, either as whole milled plant organ (following RWD processing), ethanolic extract, or purified betulinic acid were added to the core matrix to enhance anti-inflammatory and wound healing activities. Another attribute of birch bark is broad and potent antitumor activity, through activation of the mitochondrial apoptotic pathway, complementary to that of other phytochemicals in VC, RC, TC, and AC. It is cytotoxic to glioblastoma and lung, colorectal, breast, and prostate carcinoma. Betulinic acid is under investigation for its potential activity against sarcomas, a diverse group of malignancies arising from mesenchymal tissues.

In response to its potential for treating sarcoma, a dog that presented with a rare form of soft tissue sarcoma (STS), that was difficult to distinguish from osteosarcoma, was administered a simple preparation of milled, whole birch bark from an Alaska source, as described in the Case Study. Prior to onset of this trial, the birch bark had been examined for content of BA and was found to contain between 2-20% by weight with the highest concentration occurring at late winter to early spring. That concentration would deliver a meaningful amount for therapeutic effects. Despite being known to have activity against many types of tumors in vitro, nothing was known about the efficacy of betulinic acid and its metabolic precursor, betulin, against rare canine STS or osteosarcoma in vivo. A peer-reviewed article later confirmed bioactivity of both against canine osteosarcoma cell lines (Zhao, J. In Vivo. 2018, 32, 1081. doi:10.21873/invivo.11349).

### Example 7—Case Study of Canine Osteosarcoma

A 7.5-year-old, 65-pound, female retired racing greyhound was diagnosed with a rare soft tissue sarcoma (STS). A veterinarian performed annual, routine examination of the dog on Oct. 27, 2016, with only note of dry skin and administration of a scheduled rabies vaccination. On Oct. 30, 2016, owner noticed a lump on left forearm of the dog and witnessed rapid increase in size when reported to veterinarian on Oct. 31, 2016. Upon examination, appetite was reported as decreased, but temperature and routine bloodwork were normal.

Attempted fine needle aspiration of the lump found it to be very firm and impenetrable with no diagnostic cells seen on smear, only red blood cells and morphologically odd neutrophils and macrophages. Two samples of the very firm, fibrous-like, lump tissue were taken surgically with a scalpel blade and an osteocore biopsy protocol was used to take deep bone sample. By Nov. 2, 2016, the lump had doubled in size (length) from ˜1.25 cm to palpable ˜2.55 cm as a very firm, solid, immoveable mass on medial distal left radius, just proximal to carpal joint, but not involving carpal joint, normal range of motion, or causing demonstrable pain. On Nov. 9, 2016, the mass felt like bone but, on x-ray, did not appear to involve bone.

A possible small bone fragment or osteophyte/sesamoid like bone were present at base of lump. Subcutis microscopic evaluation showed sections were comprised of sheets of neoplastic mesenchymal cells within a minimal fibrovascular stroma. Neoplastic mesenchymal cells were described as elongate with moderate eosinophilic cytoplasm. There was mild-moderate anisocytosis and anisokaryosis. Nuclei were round to oval with lightly stippled chromatin and variably prominent small nucleoli.

Diagnosis was STS, grade 1. Mitotic index: 0 MF/10, 400× fields Margins: Histologic changes extend to the tissue margins. Vascular invasion: None. Recommended treatment included amputation with radiation therapy afterwards. If this is not an option, others include debulking or wait-and-see what happens, or debulking and metronomic therapy with oral cyclophosphamide; however, STS does not respond well to chemotherapy alone. Even with surgery, successful therapeutic outcome was unlikely due to poor margins at the tumor location. Veterinarian noted that owner, instead, chose palliative care, with “birch bark tea”. The actual palliative care was ˜1 g/bid of finely milled birch bark powder mix, collected near Soldotna, Alaska and fed in the dog's food beginning Nov. 1, 2016 at the time of tumor appearance, and continued for ˜10 months. As of Nov. 21, 2016, veterinary examination of the dog noted the tumor had stopped growing and routine bloodwork was normal. Follow-up examinations on Dec. 5, 2016, Jan. 24, 2017, and Feb. 17, 2017 confirmed no further tumor enlargement and overall healthy condition of the dog. By Jun. 16, 2017, the tumor length had decreased to ˜2 cm and medial distal left radius was comparable to the right at ˜8 cm circumference. Veterinary examination of dog on Sep. 18, 2017 confirmed normal health, and again on May 18, 2018 for routine vaccination and bloodwork. During this time, the tumor mass completely resolved, and the affected location appeared identical to the unaffected one. As of Feb. 28, 2022, routine veterinary examination and bloodwork of the dog was normal and without complications from previous STS diagnosis and administration of birch bark powder. The observed success of this case likely depended on treatment immediately at observation/diagnosis and a long duration of supplement administration (approximately 10 months).

On Apr. 30, 2022, the dog's owner noticed a small, palpable mass on the neck between cervical vertebrae C4 and C5. By Jun. 20, 2022, the mass increased in size and surgical removal was performed on Jun. 24, 2022. Histopathology of this tumor classified it as a rare form of osteosarcoma, likely the same cancer treated earlier, despite inconsistent diagnoses.

According to veterinary opinion, a clean margin was impossible to achieve due to tumor depth, local invasiveness, and anatomical location. Further metastasis was probable. Birch bark was not available and the dog remained untreated. By Sep. 12, 2022, the dog showed signs of internal complications and was euthanized on Sep. 14, 2022, at the age of 13.5 years, six years after initial STS/osteosarcoma diagnosis.

As a result of this study, a specific formulation in accordance with this invention comprises core matrix with the addition of birch bark extract containing betulin and betulinic acid, and purified betulinic acid, as well as artemisinin or its derivative, artemether, for treatment of canine osteosarcoma (https://www.dogcancer.com/podcast/supplements/artemisinin-for-dogs-with-cancer-dr-nancy-reese-deep-dive/). Sourced from aerial parts/inflorescences of Artemisia annua L. (sweet wormwood) or Artemisia tilesii Ledeb. (stinkweed) and subspecies, artemisinin is a sesquiterpene lactone with an unusual peroxide bridge, more specifically an endoperoxide 1,2,4-trioxane ring, that is responsible for its bioactivity.

Artemether is a more stable, methylated derivative of artemisinin. Few other natural compounds with such a peroxide bridge are known. The combination of core matrix, betulinic acid, and artemisinin have never been described for treatment of any canine cancer until it was administered first to a 13.5-year-old, 35 pound, male Australian shepherd. At diagnosis, dog presented with a large marble-sized tumor on its tail and organ involvement discovered by imaging. Given two to three months to live, the owner agreed to administer the combination with a mixture of artemisinin and artemether at a dose recommended above in the antioxidant formula for three months. Currently, dog is 14.5 years old and in generally good health. Since sarcoma (e.g., fibrosarcoma, hemangiosarcoma) is common in approximately 15% of older dogs, and osteosarcoma commonly occurs in long bones of large dog breeds, with fatality, any successful therapeutic option is highly desirable (Soft Tissue Sarcoma in Dogs-DogCancer.com).

Most tumors share some common pathways in their development and progression. Justification for adding birch extract, betulinic acid, and artemisinin to core matrix pertains to common molecular targets in each type of tumor. The WNT/β-Catenin pathway, central to STS pathogenesis, also is central to osteosarcoma and progression of other tumor types (Martinez-Font, E et al. Cancers (Basel). 2021, 13, 5521. doi:10.3390/cancers13215521). Additional signaling through P53, MAPK/ERK, and PI3K/PTEN/AKT/mTOR pathways are often dysregulated in all three tumor types (Pillozzi, S et al. Cancers (Basel). 2021, 13, 3044. doi:10.3390/cancers13123044; Marei, H E et al. Cancer Cell Int. 2021, 21, 703. doi:10.1186/s12935-021-02396-8; Eddy, K. et al. Front. Oncol. 2021, 10. doi:10.3389/fonc.2020.626129). These pathways are potential targets for developing new therapies and some drugs are already in clinical trials or approved for use.

### Finishing and Packaging

The present invention may be implemented by various means known in the trade. For example, by providing a supplement packaging system comprising: a dietary supplement formulation as described above, a blister pack with individual cavities, each cavity containing a unit dosage of the dietary supplement composition and being sealed with a UV-protective cover film to prevent degradation of light-sensitive active ingredients, the cover film being composed of a material that blocks UV radiation while allowing for easy access to the unit dosage; and the blister pack being constructed with a child-resistant mechanism to prevent unauthorized access while maintaining the bioavailability of the active ingredients.

A packaging method for preserving the bioavailability of a dietary supplement formulation may be performed by encapsulating such a dietary supplement in a capsule made from a biodegradable material that provides a controlled release of the active ingredients; then packaging the encapsulated dietary supplement composition in a container with a humidity and temperature control system to maintain optimal conditions for the preservation of the active ingredients; and equipping the container with a smart label that indicates the remaining shelf-life of the dietary supplement composition based on real-time environmental conditions.

Another method for manufacturing a phytochemical-rich dietary supplement of the present invention would include harvesting plant materials from Vaccinium, Rosa, Taraxacum, and Achillea species during peak phytochemical accumulation periods as determined by phytochemical profiling; immediately freezing the harvested plant materials to a temperature of −20 degrees Celsius or lower to arrest phytochemical degradation; thawing the frozen plant materials to a semi-frozen state and homogenizing to create a uniform puree; dehydrating the puree using a low-temperature vacuum drying technique that maintains the temperature below the thermal degradation point of the most sensitive phytochemical present; and encapsulating the dehydrated material in a protective coating that shields the phytochemicals from oxidative and hydrolytic breakdown, thereby ensuring enhanced shelf-life and bioavailability upon consumption.

The phytochemicals in a dietary supplement formulation of the present invention may be stabilized by cold-pressing a mixture of Vaccinium, Rosa, Taraxacum, and Achillea plant materials to extract phytochemical-rich juices while preventing heat-induced phytochemical loss; combining the extracted juices with a natural antioxidant matrix to form a stabilized blend; lyophilizing the stabilized blend under vacuum conditions to remove water content without exceeding a temperature of 0 degrees Celsius during the primary drying phase and 25 degrees Celsius during the secondary drying phase; and directly compressing the lyophilized blend into tablets or filling into capsules in an inert gas atmosphere to minimize exposure to oxygen and moisture, thereby preserving the phytochemical potency and extending the shelf-life of the dietary supplement.

1. A dietary supplement composition comprising:
a core matrix including a blend of plant concentrates, wherein the plant concentrates consist of Vaccinium Concentrate (VC), Rosa Concentrate (RC), Taraxacum Concentrate (TC), and Achillea Concentrate (AC);
wherein the VC, RC, TC, and AC collectively provide a complete spectrum of bioactive phytochemical classes except organosulfur compounds; and
wherein the VC is standardized to contain 4-5% by weight anthocyanins, the RC is standardized to contain more than 9% by weight phenolic acids and more than 1% by weight catechins, and the TC and AC are included to expand the representation of bioactive phytochemicals.

2. The dietary supplement composition of claim 1, further comprising:
astaxanthin in a concentration effective to enhance the antioxidant properties of the composition.

3. The dietary supplement composition of claim 1, wherein:
the VC, RC, TC, and AC are derived from plants sourced from Alaska or environmentally similar habitats.

4. A method for producing a dietary supplement composition, comprising:
obtaining Vaccinium, Rosa, Taraxacum, and Achillea plant materials from Alaska or environmentally similar habitats;
gently drying the obtained plant materials to produce concentrates that preserve the phytochemical profiles of the plant materials; blending the concentrates to form a core matrix, wherein the blend comprises VC, RC, TC, and AC; and
standardizing the VC to contain 4-5% by weight anthocyanins and the RC to contain more than 9% phenolic acids and more than 1% by weight catechins.

5. The method of claim 4, wherein:
the gentle drying is performed by refractance window drying (RWD), and the concentrates are milled to a powder form with a mesh size of 40-60.

6. A method using a dietary supplement composition of claim 1, comprising periodically administering to a mammal:
an effective amount of the dietary supplement composition;
the mammal is a human, dog, or horse; and
the administration is orally in a dosage form selected from the group consisting of capsules, tablets, powders, and liquid formulations.

7. The method of claim 6, wherein:
the dietary supplement is a composition further comprising astaxanthan i n a concentration effective to enhance the antioxidant properties of the composition;
the mammal is a human, dog, or horse needing amelioration of oxidative stress-related condition. selected from the group consisting of aging, cardiovascular diseases, diabetic complications, surgical recovery, and cancer.

8. The dietary supplement composition of claim 1, for use in anti-inflammatory treatment of a mammal, further comprising:
birch bark extract standardized to contain 7-8% by weight betulinic acid; and
quercetin in a concentration effective to enhance the anti-inflammatory properties of the composition.

9. The dietary supplement composition of claim 8, further comprising:
piperine and lecithin as bioavailability enhancers.

10. The method of claim 6, wherein the dietary supplement further comprises:
birch bark extract standardized to contain 7-8% by weight betulinic acid; and
quercetin in a concentration effective to enhance the anti-inflammatory properties of the composition, and
the mammal is a human, dog, or horse needing treatment of an inflammatory condition selected from the group consisting of chronic diseases, acute infections, injury, pain, and recovery from chemotherapy.

11. The dietary supplement composition of claim 1, for use in wound healing of a mammal, further comprising:
birch bark extract standardized to contain 7-8% by weight betulinic acid; and
quercetin.

12. The dietary supplement composition of claim 11, for use in wound-healing of a mammal, further comprising:
astaxanthin in a concentration effective to enhance the wound-healing properties of the composition.

13. A method for promoting wound healing in a mammal, comprising:
administering to the mammal an effective amount of the dietary supplement composition of claim 12.

14. The method of claim 13, wherein:
the wound healing relates to phases including hemostasis, inflammation, proliferation, and maturation.

15. The dietary supplement composition of claim 1, for use in anti-tumor therapy of a mammal, further comprising:
birch bark extract standardized to contain 7-8% by weight betulinic acid; and artemisinin or artemether in a concentration effective to enhance the anti-tumor properties of the composition.

16. A method for treating or preventing tumor growth in a mammal, comprising:
administering to the mammal an effective amount of the dietary supplement composition of claim 15.

17. The method of claim 16, wherein:
the mammal is a dog, and the tumor growth is osteosarcoma or soft tissue sarcoma.

18. The dietary supplement composition of claim 1, for use in anti-aging therapy of a mammal, further comprising:
astaxanthin in a concentration effective to enhance the anti-aging properties of the composition; and
quercetin in a concentration effective to enhance the anti-aging properties of the composition.

19. The dietary supplement composition of claim 18, further comprising:
piperine and lecithin as bioavailability enhancers.

20. A method for promoting anti-aging effects in a mammal, comprising:
periodically administering to the mammal an effective amount of the dietary supplement composition of claim 18, thereby enhancing Sirtuin1 (SIRT1) protein expression and activity.
